(19) United States
(12) Patent Application Publication
(10) Pub. No.: US 2020/0000752 A1
(43) Pub. Date: Jan. 2, 2020

(54) Method for Treating Epilepsy

(71) Applicant: Vanderbilt University, Nashville, TN (US)

(72) Inventor: Jing-Qiong Kang, Nashville, TN (US)

(21) Appl. No.: 16/316,581

(22) PCT Filed: Aug. 1, 2017
(86) PCT No.: PCT/US17/44893

§ 371 Date: Jan. 9, 2019

Related U.S. Application Data
(60) Provisional application No. 62/370,601, filed on Aug. 3, 2016.

(51) Int. Cl. A61K 31/19; A61K 31/5513; A61K 9/00; A61K 9/16

(52) U.S. Cl. CPC A61K 31/19; A61K 31/5513; A61K 9/1652; A61K 9/0056; A61K 9/0019

(57) ABSTRACT

Treatment of conditions with celastrol are disclosed herein. In particular, methods of administration of celastrol for the treatment of neurological and non-neurological disorders, including epilepsy are provided.

### RELATED APPLICATION

This application claims priority from U.S. Provisional Application Ser. No. 62/370,601, filed Aug. 3, 2016, the entire disclosure of which is incorporated herein by this reference.

### GOVERNMENT INTEREST

This invention was made with government support under grant number R01 082635 awarded by the National Institutes of Health. The government has certain rights in the invention.

### TECHNICAL FIELD

The presently-disclosed subject matter generally relates to treatment of conditions with celastrol. In particular, certain embodiments of the presently-disclosed subject matter relate to the administration of celastrol for the treatment of neurological and non-neurological disorders, including epilepsy.

### BACKGROUND

About one third of epilepsy patients do not respond to conventional treatments. This subset of patients often has intractable seizures, mental compromise and high mortality rate probably associated with uncontrolled seizures. Mounting evidence suggests that multiple intracellular signaling pathways have been altered in these severe epilepsies. Conventional antiepileptic drugs work via modulating neurotransmission and they only have anti-convulsion effect. To date there is almost no disease modifying drug or anti-epileptogenesis therapy available. Thus it is critical to find compounds that can modulate epilepsy phenotype and prevent or stop the disease progression. GABA_{A} receptor mutations are frequently associated with epilepsy with varied phenotypes.

Great effort has been directed to identify disease modifying or anti-epileptogenesis drugs¹⁴. However, there is no effective drug that has been developed. One strategy is to augment neurotrophic factors to control seizures. For example, neurotrophic factors like brain-derived neurotrophic factor (BDNF)¹⁴ and fibroblast growth factor 2 (FGF-2) have been shown to exert neuroprotective effects and they have been attempted to treat seizures. BDNF and its principal receptor target TrKB-tropomyosin-receptor-kinase B, a member of the tyrosine kinase family has been intensely investigated¹⁵. In pilocarpine-induced status epilepticus (SE)¹⁶, the dual treatment of FGF-2 and BDNF in the hippocampus 3 days after pilocarpine-induced SE attenuated hippocampal mossy fiber sprouting and reduced the frequency and severity of spontaneous seizures. Erythropoietin (EPO)-derived peptide mimetics have also been proposed to treat epilepsy because they have neuroprotective, neuroregenerative and anti-inflammatory effects¹⁷. Although EPO is a glycoprotein produced mainly in the renal cortex and acts primarily on the hematopoietic system as a cytokine to induce red blood cell production in the bone marrow. EPO is also expressed in several non-hematopoietic tissues where it acts to prevent apoptosis and inflammation due to hypoxia, toxicity and injury¹⁸. NMDA antagonist MK-801 is another neuroprotective drug that has been tested in temporal lobe epilepsy. Single dose injection MK-801 after a kainite-induced SE of 90 min was capable of preventing most of the brain damage occurring in this model¹⁹.

Another rational strategy is to reduce inflammation after brain insults. There is accumulating evidence that different types of brain insults, including SE, induce inflammatory processes in the brain that critically contribute to epileptogenesis²⁰. Various pro-inflammatory mediators are induced by SE in the brain, including cytokines such as interleukin (IL-)1β, IL-6 or TNFα, complement and cyclooxygenase-2 (COX-2), which is responsible for generation of prostaglandins from arachidonic acid²⁰. However, controversial data exist because some COX-2 inhibitor prevented neuronal damage and reduced seizure frequency while other COX-2 did not exert any disease-modifying or neuroprotective effect in an electrically-induced SE²¹.

A third rational strategy of disease modifying or anti-epileptogenesis therapy is to counteract the development of neuronal hyperexcitability after brain insults. A number of studies have shown that administration of different CNS-stimulating drugs, including the adenosine antagonist caffeine, the α2 receptor antagonist atipamezole, and the cannabinoid (CB)-1 receptor antagonist rimobanant (SR141716A) exert neuromodulatory and/or antiepileptogeneic and neuroprotective effects in epilepsy models²². It is of note that these compounds exert proconvulsant activity in normal animals, so that brain insults such as SE seem to change the pharmacology of these compounds. This also suggests that there exists molecular remodeling after brain insults, resulting in alterations in the subunit composition and expression of receptors and ion channels and, thus, in their functions and pharmacology. Furthermore, brain insults seem to induce a shift from adult to neonatal receptor and ion channel functions, indicating that epileptogenesis recapitulate ontogenesis²³. Such a shift in GABAergic response polarity from hyperpolarizing to depolarizing has been described in human epileptic neurons recorded in the subculum of hippocampal slices obtained from TLE patients²⁴. This shift is thought to be a result of increased intraneuronal CL⁻ levels, caused by increased neuronal expression of NKCC1, an inwardly directed NA⁺K⁺2Cl⁻ cotransporter that facilitates the accumulation of intracellular Cl⁻, and downregulation of KCC2, an outwardly directed K+CL⁻ cotransporter. Upregulation of NKCC1 and downregulation of KCC2 in hippocampus have been described both in TLE patients and in the kindling and pilocarpine models of TLE²⁵. Therefore, the drug that could modulate the intracellular Cl⁻ like bumetanide has been investigated and the effect is not significant up to date. Hopefully, more related compounds will be developed with high brain penetration.

It is widely acknowledged that there is an unmet need for antiepileptogenic and disease-modifying drug, although great effort has been taken as mentioned above. The major hindrance of the success includes lack of physiology-relevant animal model and good understanding of the disease mechanisms. Accordingly there remains a need in current clinical practice in the area of the treatment of epilepsy, CNS diseases such as neurodegenerative and neuroinflammation diseases, and other diseases with GABA_{A} deficiencies. Treatment with a natural product small molecule would also be of distinct advantage.

### SUMMARY

Celastrol is a pentacyclic triterpenoid and belongs in the family of quinone methides. The presently disclosed subject matter includes administering celastrol to subjects. As disclosed herein, celastrol is contemplated for use as a novel treatment that could benefit epilepsy and other neurological disorders including neurodegenerative disorders, central nervous system (CNS) disorders and brain tumors. Methods of using this compound as a novel disease-modifying drug that could be used for epilepsy as well as many other CNS diseases is also disclosed.

The presently-disclosed subject matter includes methods for treating epilepsy. In some embodiments, the methods include administering celastrol or a derivative thereof. In some embodiments, the subject has epilepsy. In some embodiments, because of its broad pharmacological effects and the pivotal roles of the compound in the central pathways in cell death and survival, effects on synaptic scaffold proteins, inflammation and heat shock protein response and proteasome degradation, the compound can be a treatment option for many diseases including but not limited to epilepsy, neurodegenerative diseases, encephalitis and even brain tumors based on different dosages. In some embodiments, the condition can be encephalitis, Alzheimer's, Parkinson's or Huntington's. In some embodiments, the treatment delays seizure onset, shortens seizure duration, or reduces seizure severity. In some embodiments, the epilepsy is selected from Dravet syndrome, primary epilepsy or secondary epilepsy.

In some embodiments, the treatment includes administering diazepam. Celastrol is, in some embodiments administered orally, intraperitoneally, or intravenously. In some embodiments, the celastrol is administered intraperitoneally in the range of 0.1 mg/kg to about 2.5 mg/kg, and in some embodiments the dosing is at about 0.1, 0.2, 0.3, or 0.5 mg/kg to about 0.6, 0.7, 0.8, 0.9, or 1 mg/kg. In some embodiments, the dosing it at about 0.3 mg/kg. In other instances, the celastrol is administered orally in the range of 1, 2, 3, 4, or 5 mg/kg to about 6, 7, 8, 9, 10, 11, 12, 13, 14, 15, 16, 17, 18, 19, or 20 mg/kg. In some embodiments, a daily oral dose is at about 5-10 mg. In some embodiments, the subject is an animal subject, celastrol is provided in an animal food product, and the administering comprises feeding the animal subject the animal food product. In some embodiments, the administering comprises intermittent dosing at about 0.1 mg/kg to about 20 mg/kg, in some embodiments, the dosing is at about 0.1, 0.2, 0.3, or 0.5 mg/kg to about 0.6, 0.7, 0.8, 0.9, or 1 mg/kg.

In some embodiments, a derivative of celastrol is administered for treatment. In some embodiments, the derivative of celastrol is selected from

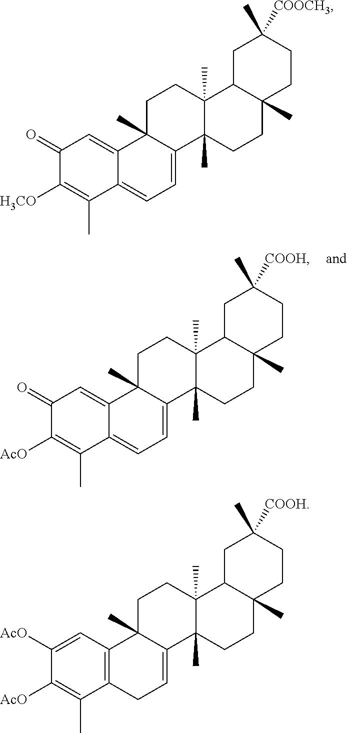 [SMILES: COC(=O)[C@]1(C)CC[C@]2(C)CC[C@]3(C)C4=CC=C5C(=CC(=O)C(OC)=C5C)[C@]4(C)CC[C@@]3(C)C2C1 ; CC(=O)OC1=C(C)C2=CC=C3[C@@](C)(CC[C@@]4(C)C5C[C@](C)(C(=O)O)CC[C@]5(C)CC[C@]34C)C2=CC1=O ; CC(=O)Oc1cc2c(c(C)c1OC(C)=O)CC=C1[C@@]2(C)CC[C@@]2(C)C3C[C@](C)(C(=O)O)CC[C@]3(C)CC[C@]12C]

In some embodiments, celastrol is modified to remove celastrol's known covalent modifying properties to obtain the celastrol derivative. In some embodiments, other deoxygenated analogs of the A-ring of celastrol are also envisioned. In some embodiments, to enhance drug permeability, celastrol is provided linked to a poly(ethylene glycol) (PEG) substituent. In some embodiments, the PEG substituent is amide linked.

In some embodiments, the celastrol is administered orally as a suspension or solution. In some embodiments, the celastrol is provided as a lipid nanoparticle suspension. In some instances, the celastrol is first milled to reduce particle size. In some instances, the celastrol is provided in a lipid excipient, such as Labrasol. In some instances, the celastrol is provided in a 20% suspension hydroxypropyl-beta-cyclodextrin (HPBCD) 80% w/v water vehicle.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1 shows A. celastrol testing in several mouse models harboring mutations associated with different epilepsy syndromes and the Alzheimer's mouse model App_{SWE}/PSEN1_{dE9} which has been reported to have increased seizure activity and seizure related mortality. Celastrol was effective in reducing seizure activity in all these mouse models; B. illustrates schematically how celastrol could increase GABAergic neurotransmission by increasing surface GABA_{A} receptors and reducing the misfolded mutant subunit inside cells.

FIG. 2 includes graphs indicating the brain and plasma concentrations (nM) of celastrol in mice via intraperitoneal injection (IP) at 0.3 mg/kg over 24 hrs or 15 days (in inset). For chronic dosing, samples were collected 2 hrs after drug administration.

FIG. 3 A provides a table summarizing the PK data from the study of 7 days IP dosing of different doses of celastrol in mice (×7 days) and a single dose study of different routes (Single). B-E. include graphs showing the mean whole blood concentrations of celastrol in male C57BL/6J mice after single daily IP doses of 0.3, 0.6 and 1.5 mg/kg for 7 consecutive days and 3 mg/kg for 5 consecutive days (B); whole blood concentrations of celastrol following a single IV dose (C) or IP dose (D) and oral dose (E).

FIG. 4 include A. Representative EEGs spike-wave-discharges (SWD) from 2 month old Gabrg2^{+/Q390X} mice in C57BL/6J background treated without or with celastrol at a series of doses by intraperitoneal injection (IP) daily for 2 weeks); B. charts results of chronic administration of celastrol either via IP or oral gavage (OG) OG (daily for 14 days) on the frequency of SWDs in Gabrg2^{+/Q390X} mice. Celastrol was dissolved with DMSO first (22.5 mg/ml) and then diluted with corn oil for IP. For OG, celastrol was administered as suspension dissolved with a 20% hydroxypropyl-beta-cyclodextrin (HPBCD) 80% water (w/v) vehicle.

FIG. 5 includes A. a schematic of the intermittent dosing regimen in mice administered celastrol (0.3 mg/kg) starting at postnatal day 7; B. charts mortality of Gabrg2^{+/Q390X} mice in C57/BL/6J background; and C. charts mortality of Scn1a^{+/-} mice in C57/BL/6J background.

FIG. 6 includes A. Representative EEGs from 9 weeks old Scn1a^{+/-}C57/BL/129 mice treated with 0.9% saline (saline), celastrol (Cel, 0.3 mg/kg), diazepam (DZP, 0.3 mg/kg)⁷, stiripentol (STP, 150 mg/kg)⁴ before pentylenetetrazole (PTZ, 50 mg/kg) injection. Saline and DZP were injected 30 min before PTZ) while STP was injected 1 hr before PTZ. The boxed region in the trace (+STP a) was expanded as the trace b. Delta (0.5-3 Hz) slowing was common in EEGs from mice treated with STP. B. The number of SWDS with duration over 1 sec was quantified after PTZ injection for 30 min. There were also multiple high voltage discharges either as single spikes or trains in Scn1a^{+/-} mice that were not quantified here (n=2 for each condition). Note: Celastrol was dosed with the regimen described in FIG. 3A. The mouse was on the 8^{th} day of drug holiday when tested. There is unlikely any celastrol in the mouse plasma to interfere PTZ absorption. Stiripentol was dosed at 150 mg/kg because mice dosed at 300 mg/kg appeared lethargic and had increased mortality.

FIG. 7 charts 15 days old cultured cortical neurons from the wild-type mouse brains (for survival) or HEK 293T cells transfected with GABAAR α1, β2 and γ2 subunits (for GABAAR expression) treated with 0, 0.125, 0.25, 0.5, 1, 2, 4 and 8 μm of celastrol for 4 hours. The neuronal viability was determined with membrane integrity by trypan blue exclusion method. Mean survival was determined by counting eight randomly selected, non-overlapping fields with each containing approximately 10-20 neurons (viable+nonviable) (n=4 different cultures). GABAAR expression was determined by the high-throughput flow cytometry. HEK 293T cells were transfected with human α1, β2 and γ2 subunit cDNAs at 1:1:1 ratio for 48 hours. The al subunit was chosen as readout. The anti-α1 antibody was directly conjugated with Alexa 488 (data are mean±SEM, n=4). The healthy population was gated.

FIG. 8 includes A. images of an eight-month old male Scn1a^{+/-} mouse with chronic intermittent dosing as sampled in both cortex and hippocampus; B. summarizes results of testing indicating normal function of liver, heart and kidney and normal total protein and metabolics; and C. includes images of Hematoxylin & Eosin (HE) staining indicating normal cell numbers, morphology and viability of liver, kidney and heart.

FIG. 9 includes proposed compounds for preparation and testing modified from the parent compound.

FIG. 10 includes data showing celastrol reduced the mutant bad protein like GABRG2(Q390X) subunits including western blots of total lysates from A. HEK 293T cells expressing wild-type α1β2γ2 (wt) or the mutant α1β2γ2(Q390X) (mut) receptors for 48 hrs or B. from 1 year old wt or Gabrg2^{+/Q390X} (het) mouse brains; C. imaging showing the het mice had γ2 subunit protein aggregates which were colocalized with active caspase 3; and D. Celastrol (1 μm) application for 4 hrs reduced the mutant γ2 (Q390X) subunit protein in HEK 293 T cells transfected with γ2(Q390X) subunit cDNAs for 48 hrs. LC stands for loading control.

FIG. 11 shows surface (A) and total (B) wild-type al subunits in the wild-type (wt) or the mutant (mut) α1β2γ2 receptors measured with high throughput flow cytometry. HEK 293T cells were transfected with wt γ2 or γ2(Q390X) subunits in combination with al and 132 subunits at 1:1:1 cDNA ratio for 48 hrs. Celastrol was applied 4 hrs before harvest. The cells were either unpermeabilized for surface staining (A,C) or permeabilized for total staining (B, D).

FIG. 12 shows celastrol increased the current amplitude in the mutant GABA_{A}α1β2γ2(Q390X) and α1β2γ2(R82Q) receptors. HEK 293T cells were transfected with the human GABA_{A} receptor α1, β2 subunits with the wild-type γ2s, the mutant γ2s(Q390X) or γ2s(R82Q) subunits for 48 hrs. Celastrol (1 μm) was applied 4 hrs before the patch clamp recordings. A. Lifted whole cells were recorded with the application of GABA 1 mM for 6 sec. Cells was voltage clamped at −50 mV. B. Celastrol (1 μm, 4 hrs) increased the current amplitude in both mutant α1β2γ2 (Q390X) and α1β2γ2(R82Q) receptors.

FIG. 13 provides A. representative traces of GABAergic mIPSCs from cortical layer VI pyramidal neurons from 2-4 month old wild-type (wt) and heterozygous (het) Gabrg2^{+/Q390X} mice untreated or treated with celastrol (0.3 mg/kg, IP) for 2 weeks; and plots of the amplitude (B) or frequency (C) of GABAergic mIPSCs in each condition.

FIG. 14 includes SDS-PAGE analysis of the surface proteins (A) and total protein of cortex (B) from the live mouse brain slices of wild-type (wt) or Gabrg2^{+/Q390X} (het) mice untreated or treated with Celastrol (0.3 mg/kg, IP) for 14 days; the protein IDVs of surface wild-type γ2 or al subunits (C) or total wild-type γ2 subunits (D) were normalized to its loading control and then to that in untreated wild-type mice which was arbitrarily taken as 1.

FIG. 15 includes SDS-PAGE analysis of the biotinylated surface proteins from either cortex (A) or thalamus (B) of wild-type (wt) or Scn1a^{+/-} (het) mice untreated or treated with celastrol (0.3 mg/kg, IP) for 14 days; C. The protein IDVs of al subunits were normalized to its loading control and then to that in either cortex or thalamus in untreated wild-type mice. D. The protein IDVs of γ2 subunits were normalized to its loading control and then to that in either cortex (cor) or thalamus (tha) in untreated wild-type mice (n==4); E. celastrol treatment increased seizure threshold and decreased seizures in the het mice after pentylenetetrazol (PTZ) injection (50 mg/kg, IP); F. Mice untreated or intermittently treated with celastrol (as detailed in FIG. 4A) were recorded for EEGs for 24 hrs; and G. includes seizures scored as blind to mouse genotype, where mice were from the same litter, and n=2 for each genotype.

FIG. 16 includes images of transfected HEK 293T cells with α1, β2 and wild-type γ2S (wt) or the mutant γ2S(Q390X) (mut) subunits treated with celastrol.

FIG. 17 includes SDS-PAGE analysis from HEK 293T cells expressing the wild-type (wt) or the mutant α1β2γ2(Q390X) (mut) receptors (A) or from Gabrg2^{+/Q309X} mouse cortex (B); C. Paraffin-embedded brain sections from 1 year old wild-type (wt) and heterozygous Gabrg2^{+/Q390X} (het) mice were stained with the active form of caspase 3 (green) and NeuN (red). The cell nuclei were stained with TO-PRO-3 (blue). In B and C, mice were treated with celastrol 0.3 mg/kg (IP) for 2 weeks.

FIG. 18 A. includes immunoblots of synaptosomes from mouse forebrains immunoblotted by rabbit polyclonal anti-γ2 subunit antibody and synaptic scaffold proteins including gephyrin, collybistin, synaptogamin 1 and neuroligin II; B. includes results for staining of mouse brains from 3-4 month old Gabrg2^{+/Q390X} mice untreated or treated with celastrol (0.3 mg/kg) for 14 days and their respective wild-type littermates, stained with rabbit anti-γ2 subunit and mouse monoclonal anti-gephyrin antibodies. The nuclei were stained with TO-PRO-3; C. charts the raw fluorescence values of gephyrin measured by ImageJ.

FIG. 19 shows A. flow chart depicting an overview of the Barnes maze; B. includes measurements of mice at 2-4 months old for Gabrg2^{+/Q390X} and C. 6-8 months old for Gabrb3^{+/-} mice showing differences in mice untreated or treated with celastrol (0.3 mg/kg, IP) for 2 weeks were trained to find the target hole which was hidden during probe trial. The total time spent at each of the 12 holes was assessed.

FIG. 20 shows celastrol increased the expression of mutant GABA_{A} α1β2γ2(R82Q) receptors associated with childhood absence epilepsy and was more effective in enhancing GABA_{A} receptor subunit expression than Stiripentol. A. includes SDS-PAGE analysis of HEK 293T cells transfected with α1, β2 and the mutant γ2 (R82Q) subunits with Celastrol (Cel) or stiripentol (Sti) applied at different concentrations 4 hrs before harvest. B. Protein IDVs of α1 or γ2 subunits were normalized to the cells without treatment (0).

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, databases, websites and other published materials referred to throughout the entire disclosure herein, unless noted otherwise, are incorporated by reference in their entirety.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

Celastrol is the maj or derivative of a traditional Chinese herb medicine, Thunder God Vine (TGV) which is the core to many traditional Chinese medicine recipes and has been used in traditional Chinese medicine for long time. Anecdotally, TGV is effective in treating epilepsy and many other chronic illnesses. However, there is no well-controlled study or the clear molecular mechanisms how the compound works.

Celastrol (tripterine) is a chemical compound isolated from the root extracts of Tripterygium wilfordii (Thunder god vine) and Celastrus regelii. Celastrol is a pentacyclic triterpenoid and belongs in the family of quinone methides. In in vitro and in vivo animal experiments, Celastrol exhibits antioxidant, anti-inflammatory, anticancer, and insecticidal activities. Its effects in humans have not been studied clinically.

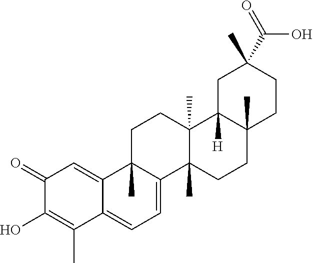

IUPAC name: 3-Hydroxy-9β,13α-dimethyl-2-oxo-24,25,26-trinoroleana-1(10),3,5,7-tetraen-29-oic acid; Scifinder/CAS number: 34157-83-0

Disclosed herein is the investigation of the use of celastrol in epileptic mouse models and that, surprisingly, celastrol administration attenuated seizure severity and improved learning and memory. Also disclosed herein are the biochemical pathways of this compound in vitro in cells expressing the mutant GABAA receptor subunits. Based on these studies and the mechanisms it targets, celastrol is contemplated for use as a novel treatment that could benefit not only epilepsy but also many other neurological disorders including neurodegenerative disorders, CNS inflammation and brain tumors. Methods of using this compound as a novel disease-modifying drug that could be used for epilepsy as well as many other CNS diseases is also disclosed.

The presently-disclosed subject matter includes methods for treating epilepsy. In some embodiments, the methods include administering celastrol. In some embodiments, the subject has epilepsy. In some embodiments, because of its broad pharmacological effects and the pivotal roles of the compound in the central pathways in cell death and survival, inflammation and heat shock protein response and proteasome degradation, the compound can be a treatment option for many diseases including but not limited to epilepsy, neurodegenerative diseases, encephalitis and even brain tumors based on different dosages.

According to one or more of the embodiments disclosed herein, celastrol has been identified as a therapeutic treatment for epilepsy, neuroprotection, reduction in seizures, improved learning and memory, and increased GABAergic neurotransmission. For example, according to data from the mouse model, celastrol treatment reduced the total amount of the mutant γ2(Q390X) subunits while the wild-type partnering subunits like al was increased. Furthermore, celastrol improved the memory in Alzheimer's disease mouse model APP/PSEN1 mice8, suggesting that celastrol can cross the blood-brain-barrier and using it as a treatment option for example, for epilepsy, is feasible. The methods of treatment disclosed herein include treatment for severe epilepsy syndromes of both acquired and genetic epilepsies, as well as neurological diseases in which celastrol targets multiple signaling pathways involved in neurological diseases, as the compound Celastrol has multiple pharmacological effects, including anti-inflammatory, antioxidant, modulation of heat shock proteins (hsps), inhibition of NF-kB pathways, neuroprotective and promotion of survival, as disclosed herein.

The presently disclosed subject matter includes treatment with celastrol for epilepsy, including primary or genetic epilepsy caused by gene mutations, and secondary or acquired epilepsy. Neurodegenerative diseases such as Alzheimers, Parkinsons's and Huntington's, brain tumors and the comorbidities like seizures, and CNS inflammation such as encephalitis are also contemplated for treatment with celastrol and the methods disclosed herein. In another embodiment, the methods include administration for the treatment of tumors.

The methods of treatment with celastrol include, in some embodiments, treatments of neurological or non-neurological disorders involving inflammation, protein misfolding and aggregation, and/or oxidative injury. In some embodiments, the methods of treatment with celastrol include improving the outcome of many diseases given its targets at the central pathways of protein metabolism, cell survival and inflammation⁶⁻⁸. For example, GABRG2(Q390X) mutation⁹ is associated with the most severe kind of epilepsy, Dravet syndrome (DS), which is also associated with many mutations in other ion channel genes like GABRA1¹⁰, SCN1A¹¹, SCN1B¹² and SCN2A¹³. Thus, the methods of treatment with celastrol disclosed herein can improve treatment in DS not only associated with GABRG2 mutations but also with other ion channel gene mutations. Celastrol may be used for other acquired epilepsies like those secondary to neurodegenerative diseases like Alzheimer's disease and inflammation. Thus, celastrol treatment could not only improve the outcome of both genetic and acquired epilepsy, it could also improve the outcome of many other neurological diseases in addition to treating seizures in those diseases. Accordingly, the invention features a method of treating a subject that has or is at risk of developing a medical condition that is amenable to treatment with celastrol.

In this regard, the terms “treatment” or “treating” refer to the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another therapy directed toward the improvement of the associated disease, pathological condition, or disorder.

In some embodiments, celastrol is administered to treat seizures. In some embodiments, the administration is prior to a seizure event, in other embodiments, the celastrol can be administered immediately after or subsequent to a seizure event. In some embodiments, the administration of celastrol delays seizure onset, shortens seizure duration, or reduces seizure severity.

In some embodiments, the celastrol is administered in intermittent dosing. In some embodiments, the celastrol can be administered as a single bolus or intermittent injections. In some embodiments, the intermittent dosing is performed by dosing once daily for some time frame followed by no administration for a time frame. In some embodiments, the time frame is from about one day to about one month. In some embodiments, the celastrol is administered at 0.1 mg/kg to about 2.5 mg/kg.

In this regard, the term “administering” is not particularly limited and refers to any method of providing a celastrol and/or pharmaceutical composition thereof to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, subcutaneous administration, intravitreous administration, intracameral (into anterior chamber) administration, subretinal administration, sub-Tenon's administration, peribulbar administration, administration via topical eye drops, and the like. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

In some embodiment, celastrol can be provided as a monotherapy. In some embodiments, celastrol can be co-administered with another composition for treatment. In some embodiments, the composition is diazepam.

As used herein, the term “subject” includes both human and animal subjects. Thus, veterinary therapeutic uses are provided in accordance with the presently disclosed subject matter.

As such, the presently disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

In this regard, in some embodiments of the presently disclosed subject matter, an animal food product is provided, comprising celastrol. In some embodiments, an animal food product is provided, comprising celastrol and diazepam. In some embodiment, a method of treating a condition with reduced GABA_{A} involves providing celastrol in an animal food product, and feeding an animal subject the animal food product, thereby treating the condition. In some embodiments, the method further involves providing celastrol and diazepam in the animal food product. In some embodiments, the condition is epilepsy, such as Dravet syndrome, primary epilepsy or secondary epilepsy.

Mutations in both sodium channel and GABA_{A} receptor (GAB_{A}R) subunit genes have been frequently associated with idiopathic generalized epilepsies (IGEs). These epilepsy syndromes vary from benign febrile seizures to severe Dravet syndrome (DS) with intractable seizures and mental decline. The underlying mechanisms of this phenotypic variability are unclear. At the single gene level, mutations in the SCN1A and GABRG2 that encodes γ2 subunits are most frequently associated with epilepsy. Missense mutations in these two genes are more likely to be associated with milder phenotypes while truncation mutations in these two genes are more likely to be associated with more severe phenotypes like DS^{27,28}.

About 80% of DS are associated with SCN1A loss of function mutations, especially truncation mutation^{29,30}. Only a few cases of DS are associated with truncation mutations in GABRG2 subunit. Evidence from multiple SCN1A genetically modified animal models³¹⁻³³ suggests that the impaired action potential and firing of GABAergic interneurons is the underlying cause of epilepsy and DS associated with SCN1A mutations. GABA_{A}Rs distribute both extra synaptically and synaptically and the number of synaptic GABA_{A}Rs correlates with inhibitory synaptic strength. Thus the underpinning mechanisms of DS associated with GABRG2 would be different than that of SCN1A. But these two different mechanisms converge on the final common pathway that gives rise to DS in both conditions. GABRG2(Q390X) mutation is associated with the DS in two independent pedigrees. We had extensively characterized this mutation in vitro^{4,5}. Our previous studies have demonstrated that the GABRG2(Q390X) mutation is not only loss of function but has dominant negative suppression on the partnering wild-type subunits⁵. In addition to the severe impairment of GABAAR channel function, the mutant GABRG2(Q390X) subunits formed SDS-insoluble high molecular mass protein complex in vitro⁴. This high protein complex was confirmed by mass spectrometry to contain the mutant subunit protein as well as wild-type GABR subunits. We have demonstrated that the mutant γ2(Q390X) protein was also accumulated and aggregated in heterozygous Gabrg2^{+/Q390X} knock-in mice. Surprisingly, this ion channel epilepsy mutant protein was identified to form protein aggregates. The mutant protein aggregation or formation of high molecular mass protein complex is a hallmark for neurodegenerative diseases³⁴⁻³⁶. But the pathologic effect of this mutant γ2(Q390X) protein aggregation in epilepsy is unclear. In the past 4-5 years, we have substantially characterized the Gabrg2^{+/Q390X} knock-in mouse model and identified the mutant protein exacerbate epilepsy phenotype. The trafficking deficient mutant protein that contributes to epilepsy and comorbidity and exacerbates the disease phenotype, causing sudden unexpected death³⁴ accumulated in the neurons and caused chronic degeneration in the mouse cortex⁹ and this could lead to a more severe epilepsy compared to those without the mutant protein accumulation³². This further supports our previous in vitro finding that the different mutant protein may have different degradation rates¹⁵. Some may have slow degradation and cause mutant protein accumulation and impose dominant negative suppression on the wild-type partnering subunits and reduce the function of the remaining wild-type subunits^{12,14}. This suggests that the production of the mutant protein like GABRG2(Q390X) subunit (bad protein) and the resulting unknown intracellular disturbance is the key to exacerbate the disease phenotype and thus presents as a good target for disease-modifying therapy for treating epilepsy.

Based on the identification that the mutant protein resulting from the loss of function mutation like GABRG2(Q390X) is toxic and exacerbates the disease phenotype, removing the mutant protein by celastrol could modify the disease phenotype. Thus, the methods of treatment disclosed herein could be potentially beneficial for multiple diseases given the central pathways of cell survival, heat shock chaperones and inflammation to which celastrol targets. Disturbed protein homeostasis has been proposed to be involved in multiple diseases involving gene mutation, protein misfolding and aging. Based on studies from multiple cell and animal models, it is likely celastrol could activate heat shock chaperones and restore protein homeostasis in many diseases. Given the broad roles of heat shock chaperones in multiple cell functions, this proposal may have enormous clinical implications for developing a disease modifying drug for multiple diseases far beyond epilepsy. However, high doses of celastrol will cause cell death. Thus, working out the proper dosing will aid in utilizing the drug for desired purposes.

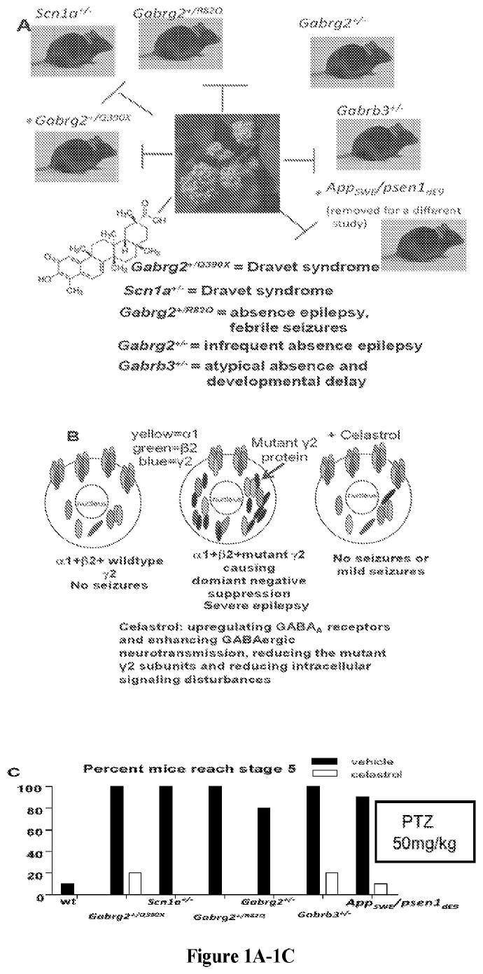

Celastrol is effective in reducing seizures and mortality in severe epilepsy mouse models with or without mutant protein aggregation. Thunder God Vine (TGV) is the core to traditional Chinese herbal medicine, and its major derivative is celastrol. Anecdotally, Chinese herbal medicine is effective for treating epilepsy but there are no well-controlled studies and the molecular mechanisms of action are not clear. However, it has been well studied that TGV is effective for treating rheumatoid arthritis, lupus and tumors due to its anti-inflammatory and anti-PI3K/AKT/ERK1/2 effect. Celastrol has been proposed to be the key to numerous therapeutic doors due to its multiple effects including stress chaperone regulation, proteasome inhibition, decreasing calcium influx, modulating PI3K-AKT/ERK1/2 pathways as well as its anti-inflammatory and antioxidant activity. The effect of celastrol has been tested in multiple cellular models for various kinds of diseases. Disclosed in the examples is the effect of celastrol in vitro in HEK 293T cells expressing the mutant GABRG2(Q390X) subunits and in vivo in Gabrg2^{+/Q390X} knock-in mice as well as other epilepsy mouse models. We have demonstrated that celastrol could upregulate GABA_{A} receptor expression and is effective in reducing seizures and improving cognition in all the tested mouse models (FIG. 1).

Additionally or alternatively, in some embodiments, a kit may be provided for treatment of epilepsy. In some embodiments, the kit includes celastrol in appropriate form and method for administering celastrol. For example, in some instances the kit would contain a syringe and celastrol in appropriate form for injection. In other instances, the kit may contain celastrol in conjunction with another composition for treatment, for example, diazepam.

The presently-disclosed subject matter is further illustrated by the following specific but non-limiting examples. The following examples may include compilations of data that are representative of data gathered at various times during the course of development and experimentation related to the present invention.

### EXAMPLES

There is virtually no disease modifying drug for severe epilepsy although great effort has been taken to discover one. A novel epilepsy mouse model Gabrg2^{+/Q390X} knock-in was developed and the pathophysiological mechanisms underpinning the severe epilepsy phenotype in this mouse model was identified as well as the overlapping mechanisms between this mouse model and other neurological diseases. The mechanism of production and chronic accumulation of the mutant protein that exacerbates epilepsy phenotypes and caused neurodegeneration in Gabrg2^{+/Q390X} mice associated with Dravet syndrome¹⁷ was identified. An extensive comparison of the molecular and behavioral alterations in two mouse models of GABRG2 loss-of-function mutations associated with epilepsy with different severities³² was conducted. Based on this discovery, a disease modifying therapy of reducing the mutant protein and restoring intracellular signaling disturbed by the mutant protein was investigated. This approach could restore or upregulate the wild-type receptor channel function, reducing neuronal and synaptic injury resulting from the production of the mutant protein.

Gabrg2^{+/Q390X} Knock-in is the Best Mouse Model to Test a Disease-Modifying Drug Working Through Protein Homeostasis in Epilepsy.

We have compared all the reported epilepsy mutations in GABA_{A} subunit genes in vitro and identified that GABRG2(Q390X) mutation could be the most representative one because of its unique pathophysiology and recapitulation of Dravet syndrome in humans¹¹. Gabrg2^{+/Q390X} knock-in mouse has provided novel insights into understanding severe epilepsy. Other in-house epilepsy mouse models will be included for comparison. For example, we have demonstrated that celastrol was effective in reducing seizures and mortality in Scn1a^{+/-} mice. This suggests the drug development of celastrol will have much broader application as loss of function SCN1A mutations account for ˜80% of Dravet syndrome in humans²³.

Demonstrated herein is that celastrol reduced seizures in two mouse models of Dravet syndrome Gabrg2^{+/Q390X} and Scn1a^{+/-} with or without mutant protein aggregation. The examples identified that celastrol could upregulate GABA_{A} receptor expression and enhance GABAergic neurotransmission in both models. Given the favorable DMPK properties disclosed herein, celastrol has application as a CNS drug, and as a disease modifying drug for severe epilepsy. Celastrol was tested both in cultured cells and in the Gabrg2^{+/Q390X} knock-in mouse model associated with epileptic encephalopathy, Dravet syndrome. Based on our previous studies, mutant GABRG2(Q390X) subunit protein (bad protein) results in loss-of-function, plus, it suppresses the function of wild-type subunit protein (good protein) and GABA_{A} receptor channel function and causes neuronal death, thus exacerbating the epilepsy phenotype. By contrast, celastrol concentration dependently reduced the mutant protein and increased the wild-type protein and channel function. More importantly, celastrol treatment reduced the seizure frequency and improved the learning and memory in Gabrg2^{+/Q390X} epilepsy mice.

Preliminary Data Shows Celastrol is Effective in Reducing Seizures

In both Gabrg2^{+/Q390X} and Scn1a^{+/-} mouse models associated with Dravet syndrome, celastrol was effective in reducing seizure activity. Without being bound by theory, the reduction in seizure activity in the epilepsy mouse models was likely by enhancing GABAergic neurotransmission via restoring protein homeostasis. (FIG. 1) Thus celastrol holds great promise to be developed into a novel compound for epilepsy and potentially beneficial for multiple diseases given the central pathways of cell survival, heat shock chaperones and inflammation to which it also targets. The diseases that could benefit from celastrol include epilepsy, neurodegenerative diseases, inflammation and tumors based on different dosages.

Based on this study, celastrol could potentially improve the outcome of many diseases given its targets at the central pathways of protein metabolism, cell survival and inflammation^{1,28,35}. For example, celastrol exhibited promise in improving memory in Alzheimer's disease model App_{SWE}/PsenI_{dE9} mice.

Additionally, we have identified that celastrol could exert neuroprotection by activating AKT signaling pathway and reduce synaptic injury by preserving synaptic scaffold proteins and anti-inflammatory effect by altering NF-KB signaling pathway likely by reducing the mutant protein. While most of the examples are focused on the effect of celastrol on GABAergic neurotransmission and neuroprotection in severe epilepsy mouse model Gabrg2^{+/Q390X} knock-in, other mouse models may be included for comparison throughout the disclosure. In this regard, an asterisk stands for the mutant protein aggregation in the neurons in related mouse models. Vehicle treated or celastrol treated mice groups (0.3 mg/kg IP daily×14 days) reached stage 5 (Racine scale) after pentylenetetrazol (PTZ, 50 mg/kg, IP) injection, as shown in FIG. 1C. Celastrol treatment reduced seizure severity in all different epilepsy mouse models as well as in the Alzheimer's disease model App_{SWE}/PsenI_{dE9} mice (n=5 mice for each group).

DMPK Data Indicate Celastrol Possesses Acceptable to Favorable Properties for a CNS Drug

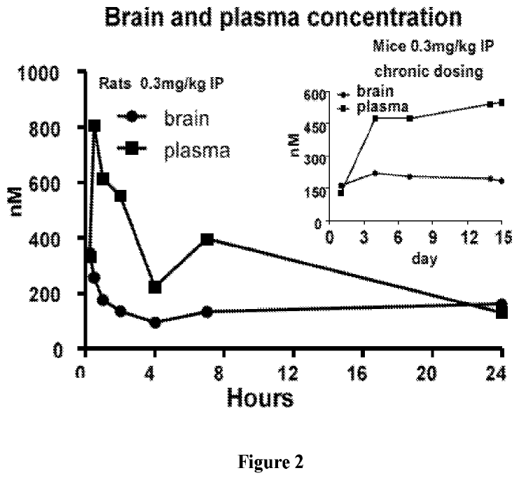

Caco-2 intrinsic permeability, MDCKII bidirectional assay, plasma protein binding, microsomal clearance, CYP450 inhibition, and in vivo exposure (IV and PO) studies were conducted, with brain and plasma concentration of celastrol after the acute and 2 weeks once-daily administration via both IP (0.3 mg/kg) and oral gavage (3 mg/kg) determined. (FIG. 2A). These preliminary data suggest that celastrol is a good candidate for CNS drug development for the following reasons: Moderate metabolic stability (hepatic microsomal CLᵢₙₜ (mL/min/kg)=31 (human), 160 (mouse); Moderate oral bioavailability and long half-life (rat F=7.8%; t_{1/2}=10 hr); High apparent membrane permeability (Pₐₚₚ≥1 in MDCKII and Caco2 cells); Low to moderate fraction unbound in plasma (fuₚₗₐₛₘₐ<0.022); High brain penetration (brain:plasma distribution >1.2 at 24 hrs); Efficacious in multiple in vivo mouse models of epilepsy; and Sustained brain exposure during chronic treatment (0.3 mg/kg, IP or 3 mg/kg oral gavage) and good efficacy via both IP and oral gavage. As provided in Table 1, Stiripentol is very potent inhibitor for 2C19 and 1A2 as highlighted in gray while the proposed dose is 150 mg to 300 mg/kg. For celastrol, the dosage proposed for epilepsy is 0.3 mg/kg with brain concentrations between 120 nM to 150 nM which is at least 15 to 80 times lower than IC50s for CYPs. Thus, there is much less a concern of CYP inhibition for celastrol than for stiripentol

TABLE 1
Cytochrome P450 (CYP) inhibition
profiles of celastrol and stiripentol

| CYPs | | | | | | | |
|---|---|---|---|---|---|---|---|
| (IC50(μM) | 3A4 | 2D6 | 2C19 | 2C9 | 2C8 | 2B6 | 1A2 |
| Celastrol | 8.94 | >10 | >10 | 4.88 | 2.19 | 3.06 | >10 |
| Stiripentol | 9.87 | >10 | 1.84 | >10 | >10 | 8.82 | 1.26 |

The PK Data of Celastrol from IV, IP and PO Studies in C57BL/6J Mice.

A. The table summarizes the PK data from the study of 7 days IP dosing of different doses of celastrol in mice (×7 days) and a single dose study of different routes (Single). B-E. The graphs show the mean whole blood concentrations of celastrol in male C57BL/6J mice after single daily IP doses of 0.3, 0.6 and 1.5 mg/kg for 7 consecutive days and 3 mg/kg for 5 consecutive days (B); whole blood concentrations of celastrol following a single IV dose (C) or IP dose (D) and oral dose (E). In A, all mice tolerated well except that the ones dosed with 3 mg/kg showed lethargy, unkempt fur and weight loss starting at day 4. In E, mice dosed with 10 mg/kg PO had similar PKs to mice dosed with 1.5 mg/kg IP.

Celastrol Administration for 14 Days Reduced Seizure Activity in Gabrg2^{+/Q390X} Knock-in Mice.

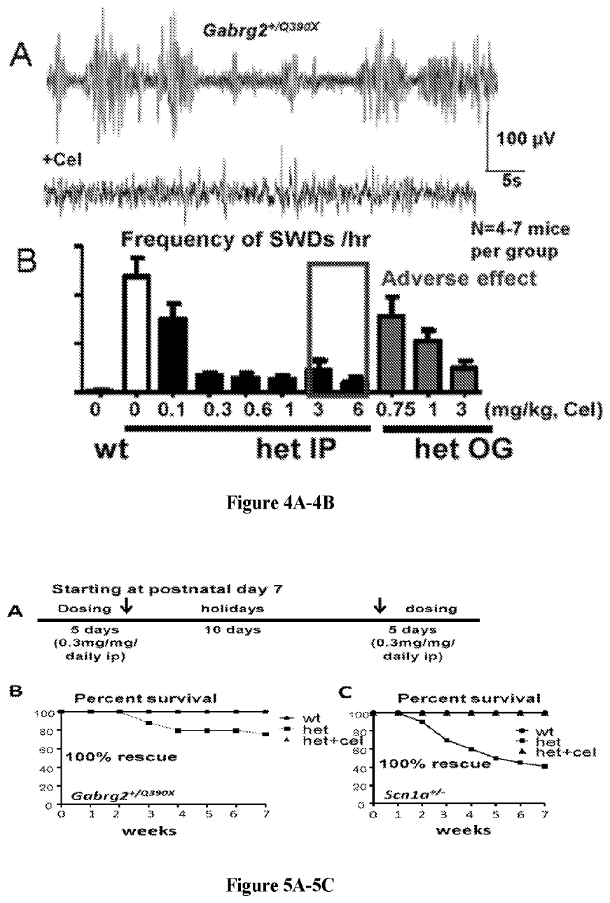

2 month old Gabrg2^{+/Q390X} mice in C57BL/6J background were treated without or with celastrol at a series of doses by intraperitoneal injection (IP) daily for 2 weeks with representative EEGs spike-wave-discharges (SWD) recorded. (FIG. 4A). Chronic administration of celastrol either via IP or oral gavage (OG) OG (daily for 14 days) dose dependently reduced the frequency of SWDs in Gabrg2^{+/Q390X} mice. (FIG. 4B). In 4B, the mice were recorded for 24 hrs for EEGs and a uniform 5-min of EEGs was scored for each hour. The SWDs with duration >1 sec were quantified. The dosing of 0.3 mg/kg (IP) daily for 14 days indicated good efficacy and tolerability, thus this dose was chosen for following studies. There was no adverse effect except in mice dosed with 3 and 6 mg/kg (IP, daily). The mice showed lethargy, unkempt fur and ˜20% to 30% of weight loss.

Early Intermittent Dosing of Celastrol.

An intermittent dosing regimen in Gabrg2^{+/Q390X} and Scn1a^{+/-} mice was used, as shown in FIG. 5A. The mice were administered celastrol (0.3 mg/kg) starting at postnatal day 7. (FIG. 5B, C). Gabrg2^{+/Q390X} mice in C57/BL/6J background had ˜25% of mortality (B) while Scn1a^{+/-} mice in C57/BL/6J background had ˜60% of mortality by week 7 (C). Early intermittent dosing of celastrol completely rescued the survival in both mouse models. The Gabrg2^{+/Q390X} mice were in C57/BL/6J background while Scn1a^{+/-} sires were from mixed S129/C57BL/6J and the dams were congenic C57/BL/6J. For each mouse model, heterozygous pups of both male and female from 6-8 litters were included. 20 heterozygous pups from each mouse line were dosed with celastrol.

Chronic Intermittent Dosing of Celastrol (0.3 mg/kg, IP) Alone had Better Efficacy in Reducing Seizure Severity than Diazepam or Stiripentol in Scn1a^{+/-} Mice

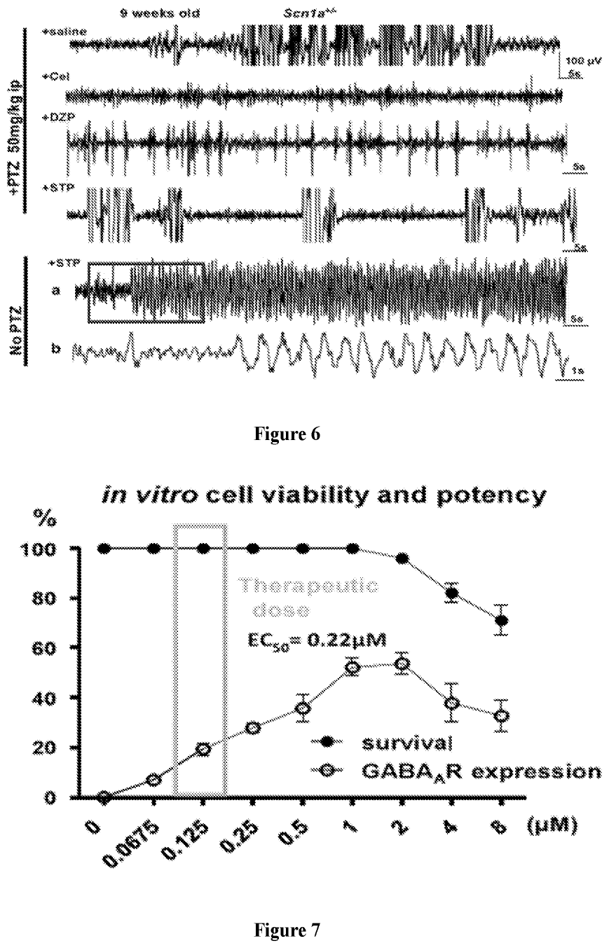

FIG. 6 includes A. Representative EEGs from 9 weeks old Scn1a^{+/-}C57/BL/129 mice treated with 0.9% saline (saline), celastrol (Cel, 0.3 mg/kg), diazepam (DZP, 0.3 mg/kg)⁷, stiripentol (STP, 150 mg/kg)⁴ before pentylenetetrazole (PTZ, 50 mg/kg) injection. Saline and DZP were injected 30 min before PTZ) while STP was injected 1 hr before PTZ. The boxed region in the trace (+STP a) was expanded as the trace b. Delta (0.5-3 Hz) slowing was common in EEGs from mice treated with STP.

TABLE 2
Number of SWDS after PTZ Injection

| | Saline | Celastrol | Diazepam | Stiripentol |
|---|---|---|---|---|
| Number SWDs | 21 | 2 | 8 | 11 |

Celastrol was dosed with the regimen described in FIG. 4A, except Stiripentol was dosed at 150 mg/kg because mice dosed at 300 mg/kg appeared lethargic and had increased mortality. The mouse was on the 8^{th} day of drug holiday when tested for SWDs. It was unlikely any celastrol in the mouse plasma to interfere PTZ absorption. As shown in Table 2, the number of SWDS with duration over 1 sec was quantified after PTZ injection for 30 min. There were also multiple high voltage discharges either as single spikes or trains in Scn1a^{+/-} mice that were not quantified here (n=2 for each condition).

In Vitro Therapeutic Dose of Celastrol has No Cellular Toxicity in Cultured Neurons.

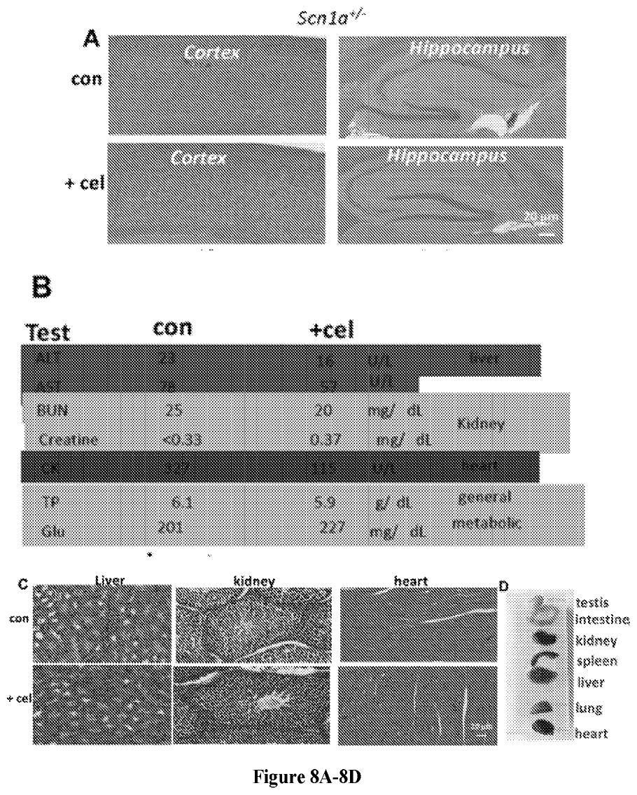

15 days old cultured cortical neurons from the wild-type mouse brains (for survival) or HEK 293T cells transfected with GABAAR α1, β2 and γ2 subunits (for GABAAR expression) were treated with 0, 0.125, 0.25, 0.5, 1, 2, 4 and 8 μm of celastrol for 4 hours. As shown in FIG. 7, Celastrol concentration dependently increased the surface al subunits but showed no reduced cell viability at the therapeutic levels. The neuronal viability was determined with membrane integrity by trypan blue exclusion method¹². The mean survival was determined by counting eight randomly selected, non-overlapping fields with each containing approximately 10-20 neurons (viable+nonviable) (n=4 different cultures). GABAAR expression was determined by the high-throughput flow cytometry. HEK 293T cells were transfected with human α1, β2 and γ2 subunit cDNAs at 1:1:1 ratio for 48 hours. The α1 subunit was chosen as readout. The anti-α1 antibody was directly conjugated with Alexa 488 (data are mean±SEM, n=4). The healthy population was gated. The box in FIG. 7 indicates the celastrol concentration in the brain of mice showed good efficacy. It was demonstrated that ˜20% to 30% increase of GABAAR substantially reduce epilepsy severity in both Gabrg2+/Q390X and Scn1a^{+/-} mice which is very achievable with celastrol 120-150 nm in the mouse brain.

Chronic Intermittent Dosing of Celastrol had Good Efficacy without Toxicity in Mice.

An eight-month old male Scn1a^{+/-} mouse with chronic intermittent dosing was investigated for cortex and hippocampus neuronal survival, which was normal (FIG. 8A). The mouse had normal blood counts (data not shown), normal function of liver, heart and kidney and normal total protein and metabolics (FIG. 8B). Normal cell numbers, morphology and viability of liver, kidney and heart are indicated by Hematoxylin & Eosin (HE) staining (FIG. 8C). Other major organs including lung, spleen, intestine, testis and leg muscle and skin were also examined with HE staining and no abnormalities were identified. The mouse had been used for breeding and 3 litters with 6-8 grossly normal pups were born, indicating normal fertility of the treated mouse and no teratogenicity of celastrol.

Creating New Compounds to Reduce Toxicity and Enhance Bioavailability.

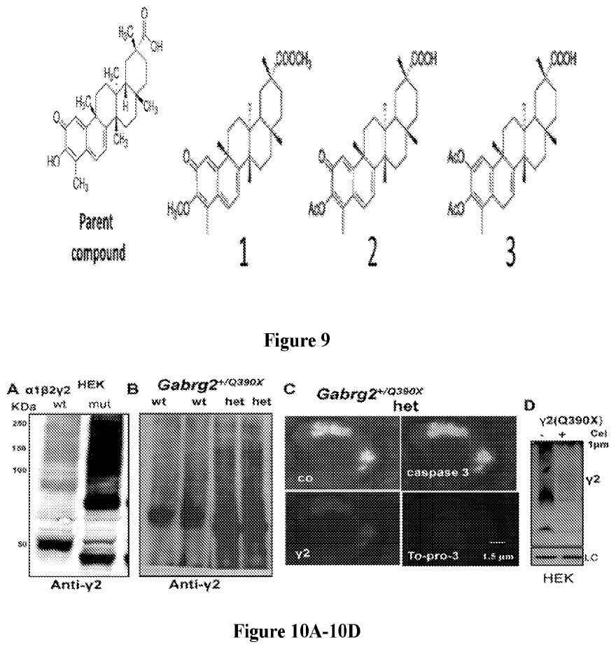

It is proposed to prepare and test three compounds modified from parent compound as shown in FIG. 9. Compound 3 lacks the methide structure and thus may help reduce toxicity. Although there is no guarantee, there is a strategy to remove celastrol's known covalent modifying properties. Other deoxygenated analogs of the A-ring of celastrol are also envisioned; however, at this time it is proposed to start with these compounds since they are well characterized, and compound 3 specifically has been shown to be devoid of covalent modification¹⁹. To enhance drug permeability, an amide linked poly(ethylene glycol) (PEG) was introduced which showed complete retention of biological activity (heat shock response was chosen as readout) compared to parent compound in a previous study.

GABRG2(Q390X) Mutation Associated with Severe Epilepsy Resulted in the Accumulation of the Mutant Subunits (Bad Protein) which Exacerbate Disease Phenotype. Celastrol could Remove the Mutant Bad Protein and Improve the Disease Outcome.

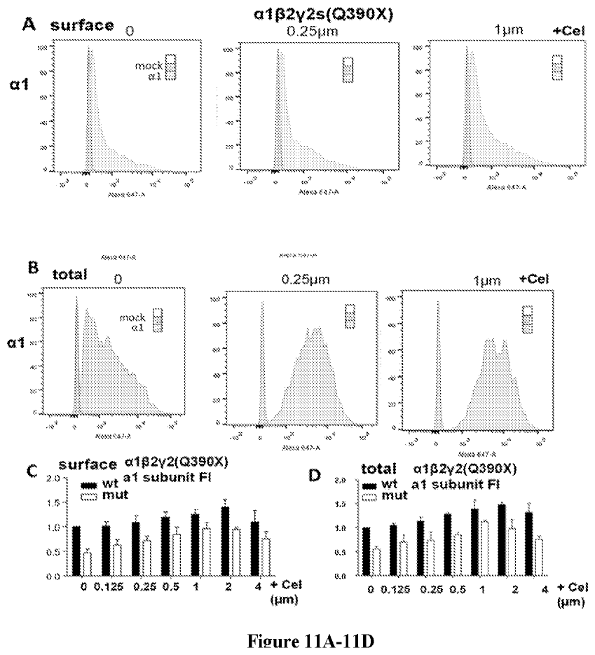

Total lysates from HEK 293T cells expressing wild-type α1β2γ2 (wt) or the mutant α1β2γ2(Q390X) (mut) receptors for 48 hrs from or from 1 year old wt or Gabrg2^{+/Q390X} (het) mouse brains were analyzed by western blot (FIGS. 10A and 10B). As shown in FIG. 10C, the het mice had γ2 subunit protein aggregates which were colocalized with active caspase 3, where To-pro is a marker for staining nuclei. Celastrol was applied for 4 hours at 1 μm, which reduced the mutant γ2 (Q390X) subunit protein in HEK 293 T cells transfected with γ2(Q390X) subunit cDNAs for 48 hrs (FIG. 10D).

GABRG2(Q390X) Mutation Associated with Severe Epilepsy in Humans Reduced the Wild-Type Subunit Protein (Good Protein) while Celastrol Administration Concentration Dependently Increased the Wild-Type Subunit Protein at Total and Surface Levels.

HEK 293T cells were transfected with wt γ2 or γ2(Q390X) subunits in combination with al and 132 subunits at 1:1:1 cDNA ratio for 48 hrs. Celastrol was applied 4 hrs before harvest. The cells were either unpermeabilized for surface staining (FIG. 11A, 11C) or permeabilized for total staining (FIG. 11B, 11D). al subunits were probed with mouse anti-α1 subunit antibody conjugated with Alexa 647. The relative al subunit fluorescence intensity (FI) was normalized to the wild-type without celastrol treatment. Celastrol concentration dependently increased the surface and total al subunits. Celastrol was applied at 0, 0.125, 0.25, 0.5, 1, 2 and 4 μm. Cell death was observed in dishes applied with 2 and 4 μm of celastrol. Thus, celastrol (1 μm) was used for all other in vitro experiments with a single concentration.

GABRG2 Epilepsy Mutations Reduced the Receptor Channel Current Amplitude while Celastrol Administration (1 μM) Increased the Mutant Channel Amplitudes.

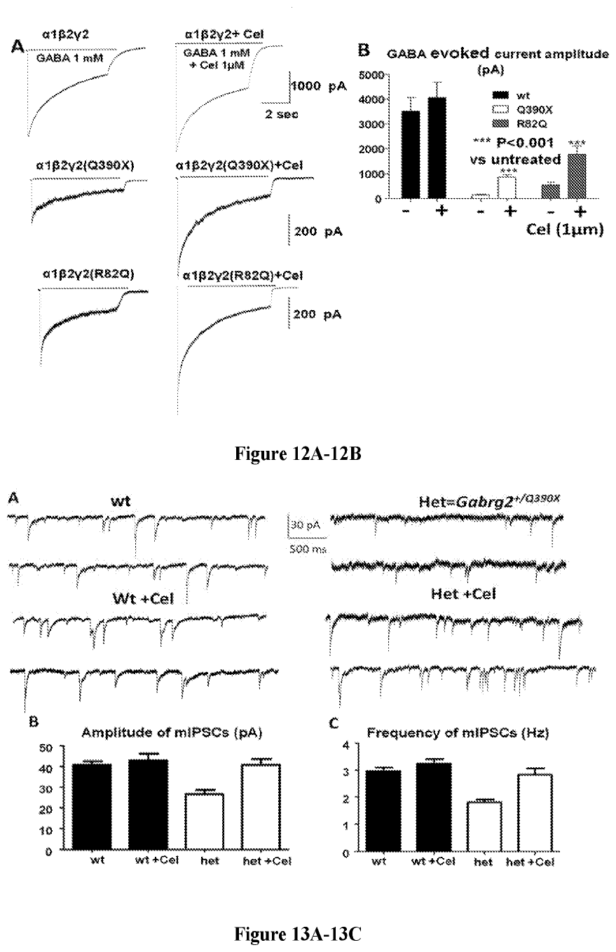

HEK 293T cells were transfected with the human GABA_{A} receptor α1, β2 subunits with the wild-type γ2s, the mutant γ2s(Q390X) or γ2s(R82Q) subunits for 48 hrs. Celastrol (1 μm) was applied 4 hrs before the patch clamp recordings. As shown in FIG. 12, the current amplitude in the mutant GABA_{A} α1β2γ2(Q390X) and α1β2γ2(R82Q) receptors were increased with celastrol application.

Celastrol Upregulated the Wild-Type GABA_{A} Receptor Expression and Increased GABAergic Neurotransmission and was Effective in Reducing Seizures in Dravet Syndrome Mouse Models of Both GABRG2 and SCN1A Mutations.

MDCK II bidirectional and Caco2 assays indicate celastrol has high membrane permeability with no significant efflux (Pₐₚₚ≥1). Furthermore, celastrol has been reported to improve memory in Alzheimer's disease mouse model App_{SWE}/PsenI_{dE9} mice¹. This suggests that celastrol is CNS penetrant, and using it as a treatment option for epilepsy is feasible.

Celastrol (0.3 mg/kg, IP) Treatment Increased GABAergic mIPSCs in the Gabrg2^{+/Q390X} Mice.

As shown in FIG. 13, celastrol administration (0.3 mg/kg, IP) for 14 days increased GABAergic neurotransmission in Gabrg2^{+/Q390X} mice. Representative traces of GABAergic mIPSCs from cortical layer VI pyramidal neurons from 2-4 month old wild-type (wt) and heterozygous (het) Gabrg2^{+/Q390X} mice untreated or treated with celastrol (0.3 mg/kg, IP) for 2 weeks is shown in FIG. 13A. The treatment increased both the amplitude and frequency of GABAergic mIPSCs, as is shown in FIGS. 13B and C, respectively. Celastrol administration increased both the amplitude and frequency of GABAergic mIPSCs in the Gabrg2^{+/Q390X} mice. This effect is consistent with its upregulation of GABA_{A} receptor subunits. There is no difference between the decay tau (time constant) in the condition treated with celastrol vs non treated. (n=7-9 cells from 3 mice in each group, *P<0.05, ** P<0.01 vs het).

Celastrol Treatment (0.3 mg/kg, IP) Increased the Surface and Total GABA_{A} Receptor Subunit Expression in the Gabrg2^{+/Q390X} Mice.

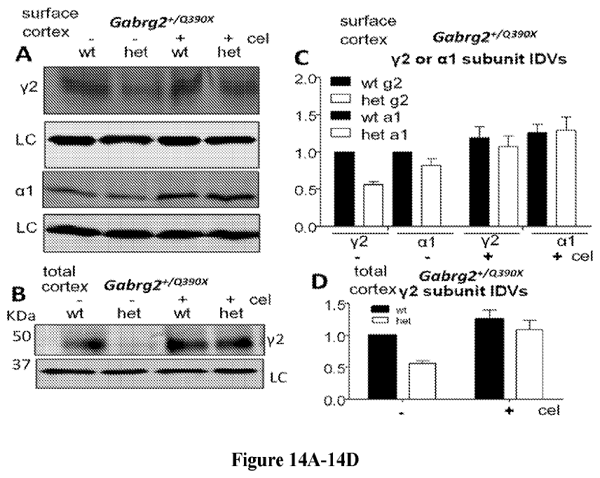

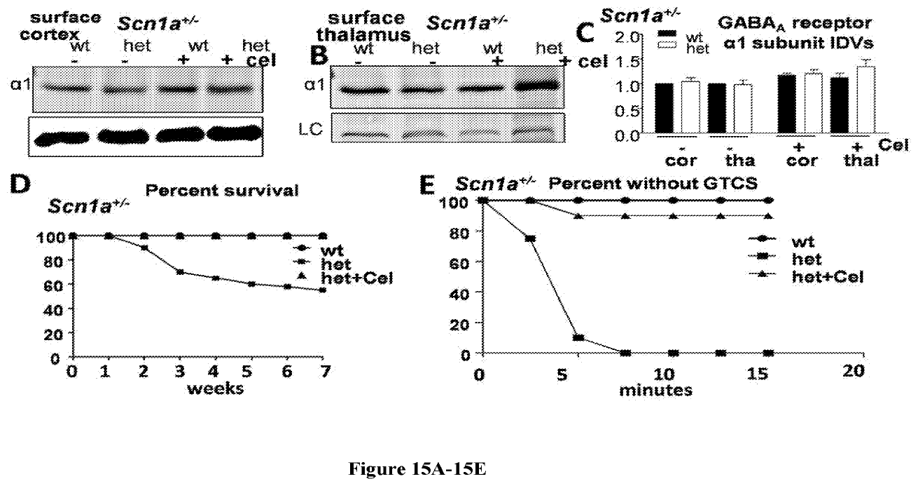

Wild-type (wt) or Gabrg2^{+/Q390X} (het) mice were untreated or treated with Celastrol (0.3 mg/kg, IP) for 14 days. As shown in FIG. 14A upper panel, the surface proteins (FIG. 14A) from the live mouse brain slices were biotinylated and analyzed by SDS-PAGE and immunoblotted with anti-γ2 or anti-al subunit antibody. In the lower panel, the protein from total lysates of cortex was analyzed by SDS-PAGE and immunoblotted with anti-γ2 subunit antibody. LC is the loading control GAPDH in the blots. FIG. 14B charts the protein IDVs of surface wild-type γ2 or al subunits (upper panel) or total level wild-type γ2 subunits (lower panel) were normalized to its loading control and then to that in untreated wild-type mice which was arbitrarily taken as 1. The increase of both the surface and total α1 and γ2 subunits were greater in the mutant mice than in the wildtype (n=4 mice).

Celastrol Treatment Increased (0.3 mg/kg, IP) the Surface GABA_{A} Receptor Subunit Expression and Reduced Seizures in Another Severe Epilepsy Mouse Model, the Scn1a^{+/-} Mice.

Wild-type (wt) or Scn1a^{+/-} (het) mice were untreated or treated with celastrol (0.3 mg/kg, IP) for 14 days, and tested at day 15. The biotinylated surface proteins from either cortex (FIG. 15A) or thalamus (FIG. 15B) were analyzed by SDS-PAGE and immunoblotted with anti-GABA_{A} receptor al subunit antibody. The protein IDVs of al subunits were normalized to its loading control and then to that in either cortex (cor) or thalamus (tha) in untreated wild-type mice (n==4) (FIGS. 15C and 15D). Celastrol treatment increased seizure threshold and decreased seizures in the het mice after pentylenetetrazol (PTZ) injection (50 mg/kg, IP), as shown in FIG. 15E. Mice untreated or intermittently treated with celastrol (as detailed in FIG. 4A) were recorded for EEGs for 24 hrs, shown in FIG. 15F. FIG. 15G includes seizures scored as blind to mouse genotype. In FIGS. 15E and 15G, MJ stands for myoclonic jerks with behavioral correlation while GTCS for generalized tonic clonic seizures. The number of myoclonic jerks (MJ) and generalized tonic clonic seizures (GTCS) is the total number over 24 hrs. In G, mice were from the same litter, n=2 for each genotype.

Celastrol Increased the Expression of Heat Shock Protein Hsp70 as Measured by High Throughput Flow Cytometry.

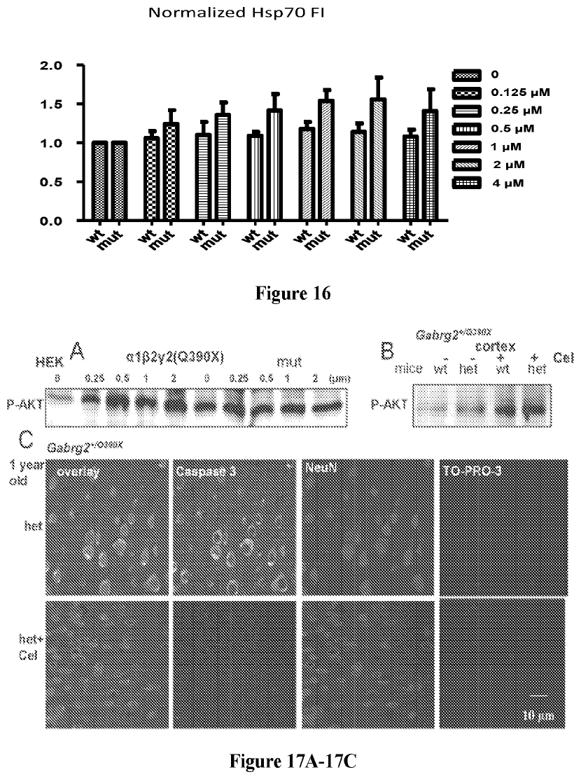

HEK 293T cells were transfected with α1, β2 and wild-type γ2S (wt) or the mutant γ2S(Q390X) (mut) subunits for 48 hrs (FIG. 16). Celastrol was applied to the cells for 4 hrs before harvest. The cells were permeabilized and stained with monoclonal hsp70 (1:200) which was then conjugated with Alexa 647. The fluorescence intensity in the celastrol treated groups was normalized to the cells expressing the wild-type (wt) or the mutant (mut) receptors without celastrol treatment (0).

Celastrol Treatment (0.3 mg/kg, IP) was Neuroprotective by Activating AKT and Reducing Active Caspase 3 in Cells and in Gabrg2^{+/Q390X} Knock-in Mice.

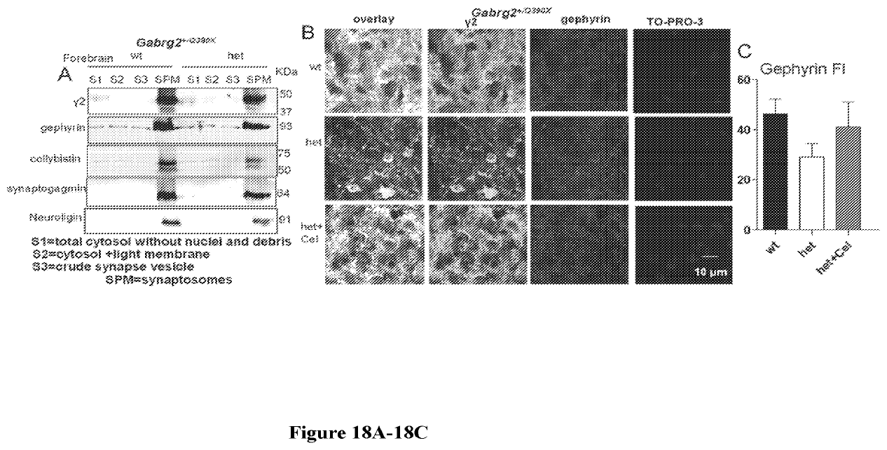

Total lysates from HEK 293T cells expressing the wild-type (wt) or the mutant α1β2γ2(Q390X) (mut) receptors (FIG. 17A) or from Gabrg2^{+/Q390X} mouse cortex (17B) were analyzed by SDS-PAGE. The membranes were immunoblotted with the phosphorylated AKT (P-AKT). FIG. 17C. Paraffin-embedded brain sections from 1 year old wild-type (wt) and heterozygous Gabrg2^{+/Q390X} (het) mice were stained with the active form of caspase 3 (green) and NeuN (red). The cell nuclei were stained with TO-PRO-3 (blue). In B and C, mice were treated with celastrol 0.3 mg/kg (IP) for 2 weeks.

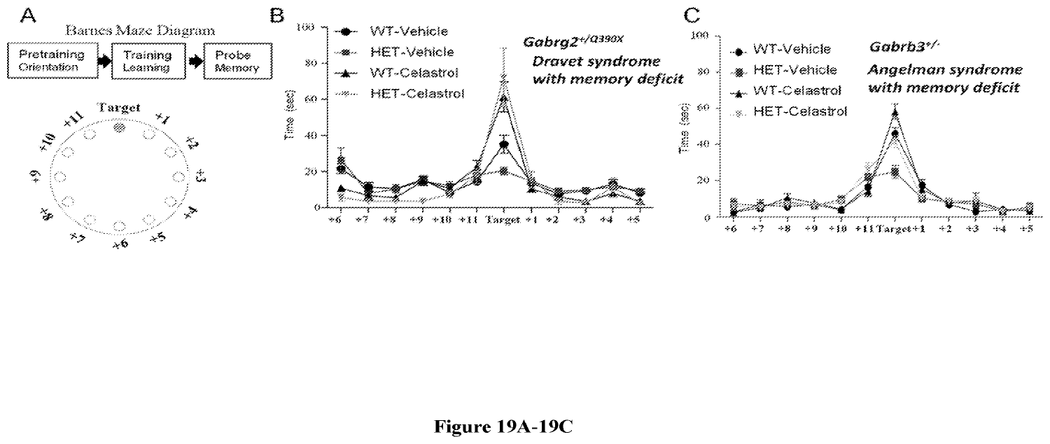

Celastrol (0.3 mg/kg, IP) rescued synaptic scaffold protein gephyrin in Gabrg2^{+/Q390X} mice. Synaptosomes from mouse forebrains were isolated by subcellular fractionation⁷. The samples were then fractionated by SDS-PAGE and immunoblotted by rabbit polyclonal anti-γ2 subunit antibody and synaptic scaffold proteins including gephyrin, collybistin, synaptogamin 1 and neuroligin II. The γ2 subunit protein and synaptic scaffold proteins were reduced in the Gabrg2^{+/Q390X} (het) mice. (FIG. 18A). Mouse brains from 3-4 month old Gabrg2^{+/Q390X} mice untreated or treated with celastrol (0.3 mg/kg) for 14 days and their respective wild-type littermates were short-fixed (30 min exposure to 4% paraformaldehyde) and sectioned on a cryostat at 15 to 30 μm. The sections were then stained with rabbit anti-γ2 subunit (green) and mouse monoclonal anti-gephyrin (red) antibodies. The nuclei were stained with TO-PRO-3. (FIG. 18B). The raw fluorescence values of gephyrin was measured by ImageJ. Celastrol treatment increased gephyrin puncta in the Gabrg2^{+/Q390X} mice, suggesting that it could also rescue other synaptic scaffold proteins. (FIG. 18C).

Celastrol Treatment (0.3 mg/kg, IP) Improved Learning and Memory in Gabrg2^{+/Q390X} and Gabrb3^{+/-} Mice.

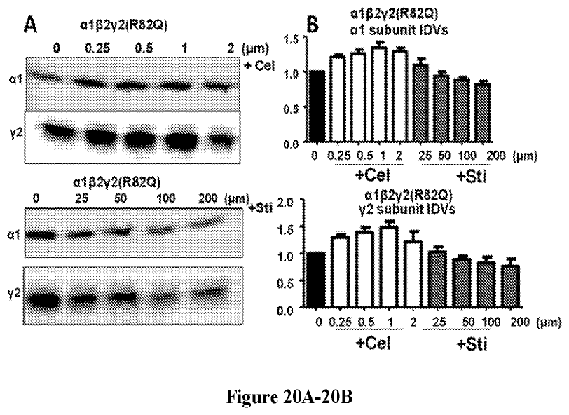

Flow chart in FIG. 13A depicts an overview of the Barnes maze. In training trials which are considered as learning test, time spent to locate the target hole was recorded and quantified for each day in each mouse genotype. In probe trials which are considered as memory test, an hour after the last training trial, each mouse was allotted a 300 sec session to find the target hole. Mice that were 2-4 months old for Gabrg2^{+/Q390X} (FIG. 19B) and 6-8 months old for Gabrb3^{+/-} mice (FIG. 19C) were untreated or treated with celastrol (0.3 mg/kg, IP) for 2 weeks were trained to find the target hole which was hidden during probe trial. The total time spent at each of the 12 holes was assessed. Both the wild-type and mutant mice spent more time in the target hole area, suggesting enhanced memory.

Celastrol could Potentially have Broader Application than Stiripentol for Epilepsy Because it Rescues Other Mutant GABA_{A} Receptors.

Celastrol increased the expression of mutant GABA_{A} α1β2γ2(R82Q) receptors associated with childhood absence epilepsy and was more effective in enhancing GABA_{A} receptor subunit expression than Stiripentol. HEK 293T cells were transfected with α1, 132 and the mutant γ2(R82Q) subunits at 1:1:1 cDNA ratio for 48 hrs. Celastrol (Cel) or stiripentol (Sti) at different concentrations was applied 4 hrs before harvest. (FIG. 20A). Total cell lysates were analyzed by SDS-PAGE and the membrane was immunoblotted against al or γ2 subunits. (FIG. 20A). Protein IDVs of al or γ2 subunits were normalized to the cells without treatment (0) (FIG. 20B). This suggests that celastrol could also be used in other epilepsy in addition to Dravet syndrome.

The effect of celastrol with stiripentol was investigated both in vitro in HEK 293T cells and in vivo in mice. Stiripentol has been proposed to be the most effective drug for Dravet syndrome. The proposed mechanisms of stiripentol include but not limited to increasing GABA transmission, inhibiting lactate dehydrogenase and improving the effectiveness of many other anticonvulsants and slowing the drug's metabolism, increasing blood plasma levels. The effect of stiripentol on GABA_{A} receptor expression and on seizure activity and in Gabrg2^{+/Q390X} mice and also in Scn1a^{+/-} mice has been extensively characterized by our research group. Celastrol has potential to be a better drug for epilepsy than stiripentol because of four reasons: celastrol could more effectively enhance GABAergic neurotransmission by upregulating GABA_{A} receptors; celastrol could potentially be used both as monotherapy and as adjunct therapy; celastrol could protect against neuronal death and synaptic injury and improve comorbidities like enhancing learning and memory; and celastrol could have much broader application than stiripentol.

Overall Methodology/Analyses

Mice.

The Gabrg2^{+/Q390X} knock-in mouse was generated in collaboration with Dr. Siu-Pok Yee at University Connecticut Health Center as previously described. Scn1a^{+/-} mouse line²³ were kindly provided by a former colleague Dr. Jennifer Keamey who is now in Northwestern University. Scn1a^{+/-} knock-out mice was in maintained in S129/SvJ background and bred into C57BL/6J F2 for experiment. Gabrg2^{+/-} knock-out, Gabrb3^{+/-} knock-out and Gabrg2^{+/R82Q} knock-in mouse lines were originally purchased from Jackson laboratory and have also been bred into C57BL/6J background for 8 generations.

GABA_{A} Receptor Subunit cDNA Plasmids:

The cDNAs encoding human GABA_{A} receptor subunits α1, β2, γ2S subunits were constructed as described previously¹⁰.

Lc-Ms-Ms System.

Protocol utilized as in the previous study²⁷.

ITRAQ/SILAC:

Protocols established for both techniques in the proteomics core are as previously described²⁶. iTRAQ (isobaric Tagging for Relative and Absolute Quantification) to measure changes in proteins in the somatosensory cortex of the wild-type and the mutant Gabrg2^{+/Q390X} mice treated with vehicle vs with Celastrol. SILAC: SILAC (stable isotope labeling by amino acids in cell culture) will be used to profile the biochemical changes in HEK 293T cells expressing the wild-type γ2 and the mutant γ2(Q390X) subunits. ITRAQ/SILAC will be used for profiling broad biochemical changes with Celastrol and Stiripentol treatment.

Brain Slice Preparation and Recording:

Coronal (300 m) or horizontal (400 m thick) brain slices containing thalamic neurons in nucleus reticularis thalami (nRT), ventrobasal nucleus (VBn), Ventral lateral thalamus (VL) and cortex will be sectioned with a vibratome in ice-cooled solution containing (in mM) 214 Sucrose, 2.5 KCl, 1.25 NaH₂PO₄, 0.5 CaCl₂), 10 MgSO₄, 24 NaHCO₃, and 11 D-glucose, pH 7.4 bubbled with 95% O₂/5% CO₂ at 4° C. Slices are then incubated in oxygenated artificial cerebrospinal fluid (ACSF)⁴⁰ at 36° C. for 30 min (Moyer and Brown, 1998). After this, slices will be kept at room temperature for at least 1 hr before recording on a Nikon Eclipse FN-1 IR-DIC microscope at room temperature. Pipette internal solution will contain (in mM): 135 CsCl, 10 EGTA, 10 HEPES, 5 ATP-Mg, and QX-314 (5 mM) (pH 7.25, 290-295 mOsm), and resistances will be of 2-4 MΩ (25). Tetrodotoxin (TIX) 1 μm will be added to the external solution. We will record the neurons in layer V-VI in somatosemsory cortex in this application. The experimental details have been described before³⁹.

Brain Slice Immunohistochemistry.

Protocols for short-fixed tissues and paraffin-embedded brain tissues¹⁷. For short-fixed tissues, the brain will be blocked and exposed to 4% paraformaldehyde for 30 min. For paraffin-embedded tissues, mice will be transcardially perfused using a fixative of 2% paraformaldehyde, 2% glutaraldehyde, and 0.2% picric acid in 0.1 M sodium phosphate, pH 7.2, and the brains postfixed in 4% paraformaldehyde overnight at 4° C.

Subcellular Fractionation and Isolation of Synaptosomes:

The procedures of subcellular fractionation were modified from a previous study for synaptosome preparation ^{18,19} The synaptosome layer (spm) was at the 1.0/1.2 M sucrose interface. To prepare postsynaptic densities, the spm fraction was diluted to 0.32 M sucrose by adding 2.5×vol of 4 mM Hepes (pH 7.4) and balanced with Hepes buffered sucrose (HBS). The diluted spm preparation was then centrifuged at 150,000 g for 30 min (TH 641:29,600 rpm). After centrifugation, the pellet was collected and suspended by adding 4 ml 0.5% triton-100 solution containing 50 mM Hepes, 2 mM ethylenediaminetetraacetic acid (EDTA) and protease inhibitors rotated for 15 min.

Synchronized EEG Recordings and Analysis.

EEG recordings have been routinely conducted for 4 years with optimized surgical procedure and recording system¹⁹. Synchronized video EEGs will be recorded from at least 8 weeks to 2 months old C57BL/6J mice one week after electrode implantation. Video-EEG monitoring will be lasting for 24-48 hrs to a week depending on the seizure frequency. Mice will be recorded continuously up to a month in the case with chronic Celastrol treatment or when seizure activity is rarely observed. During EEG recordings, mice will be freely moving with a low torque commutator (Dragonfly Inc). Mouse behaviors such as behavioral arrest during the EEG discharges will be identified to determine if mice exhibit absence seizures or other seizure types. Average seizure frequency will be determined by analyzing at least 24 hours of EEG recordings. The experimental details have been described in previous study². Analysis: A blinded reviewer analyzed the EEG off line and identified spike-wave discharges (SWDs) using criteria established for the analysis of rat models of absence epilepsy¹. Briefly SWDs were defined as trains (>1 s) of rhythmic biphasic spikes, with a voltage at least twofold higher than baseline and that were associated with after going slow waves. The reviewer quantified the SWD incidence and duration in uniform 5-min samples each hour for at least 24 hrs (12 hours for daytime and 12 hours for night). To determine if SWDs were associated with behavioral arrest, manifestations of absence seizures, we determined whether the longer SWDs (>2 s) were associated with attenuation of the EMG signal and behavioral changes on video. Because mouse movements produce slow (1-4 Hz) EMG waveforms, we will also objectively quantify the effects of SWDs on movement by measuring the relative EMG spectral power (1-4 Hz delta power)³. Average seizure frequency will be determined by analyzing at least 24 hours of EEG recordings. For EEGs during seizure induction, total 30 min of recordings after Pentylenetetrazol (PTZ) injection will be scored. The percent of mortality, number of mice reaching stage 5, the number of myoclonic jerks and SWDs will be measured.

Discussion:

Celastrol upregulated the wild-type GABA_{A} receptor expression and increased GABAergic neurotransmission and was effective in reducing seizures in Dravet syndrome mouse models of both GABRG2 and SCN1A mutations. We have demonstrated that celastrol is effective in reducing seizures in multiple epilepsy mouse models and had sustained brain concentrations (FIG. 2). The effect has been demonstrated in cells and in both Dravet syndrome mouse models Gabrg2^{+/Q390X} and Scn1a^{+/-} with or without mutant protein aggregation (FIG. 10-15). The DMPK data indicate celastrol possesses multiple favorable pharmaceutical properties for a CNS drug. The safety margin is 5 folds of the efficacious dose and the bioavailability is 20%. It indicates good efficacy in both Gabrg2^{+/Q390X} and Scn1a^{+/-} mice as it reduced seizures and mortality and improved cognition. Although there are multiple mechanisms of actions, the efficacy in seizure control and cognition improvement is likely via enhancing GABAergic neurotransmission and reducing neuronal/synaptic injury. It is clear that celastrol increases both the surface and total GABA_{A} receptor subunits. Because we used the low dose and intermittent dosing regimen was used, previously reported toxicity and side effects with doses for tumor treatment is likely not related to this study.

Prospective Study of DMPK and Celastrol Potency

Completion of in vitro DMPK and in vitro potency of Celastrol in cell based assays will include a) cellular potency for at least one biochemical pathway consistent with orally delivered drugs, EC₅₀ or IC₅₀<10 microM b) Demonstration that as a lipophilic acid (e.g. NSAIDs, third generation antihistamines, montelukast), celastrol has in vitro permeability indicative of potential for an oral therapy (Pₐₚₚ>1×10⁻⁶ cm/s) and profiling the effect of celastrol on cytochrome P450 enzymes (CYPs); c) identification of optimized formulation for improved bioavailability from oral administration.

Experimental Methodology and Analysis:

Intrinsic clearance and predicted hepatic clearance: Intrinsic clearance will be determined using the substrate depletion approach. Reactions (0.3 mL) will be conducted using a Tecan EVO (San Jose, Calif.) in 96-well polypropylene cluster tubes with a temperature controlled water-jacketed aluminum plate holder held at 37° C. Incubations will be performed in triplicate in 0.1 M potassium phosphate buffer (pH 7.4), 3 mM MgCl₂, 1 μM tizanidine, and 1 mg/mL liver microsomes. After incubation mixtures are equilibrated for 3 min at 37° C., reactions will be initiated with 10 mM NADPH to give a final NADPH concentration of 1 mM. At 0, 3, 7, 15, 25, and 45 min, a 25 μL aliquot will be removed from the reactions and delivered into 125 μL of acetonitrile containing an internal standard. The quench plates will then be centrifuged at 3,000×g for 10 min and an aliquot of the supernatants added to an injection plate with an equal volume of water. Samples will then be analyzed using LC/MS/MS.

Intrinsic clearance is calculated as:

$${CL}_{int}^{′} mL \text{/} min \text{/} kg = 0.693 \times \frac{1}{{t}_{1 / 2 ( min )}} \times \frac{1 mL}{0.5 mg of {protein}_{mic}} \times \frac{45 mg {protein}_{mic} }{1 g Liver weight} \times \frac{( A ) g of Liver weight }{kg of body weight}$$

Species-specific parameters for mouse and human liver protein content will be used. The predicted hepatic clearance will then be estimated using species-specific liver blood flow values:

$${CL}_{HEP} mL \text{/} min \text{/} kg = \frac{{Q}_{H} \times {CL}_{int}}{{Q}_{H} + {CL}_{int}}$$

Cellular Permeability: Caco2 and MDCKII Bi-Directional Assay.

Caco-2 permeability will be assessed in triplicate experiments with cell monolayers grown in multi-well collagen-coated insert plates. After confirming transepithelial electrical resistance of cells in Hank's Balanced Salt Solution (1500-2200Ω), fresh buffer containing celastrol or propranolol positive control at 2 microM (Pₐₚₚ=2−10×10⁻⁶ cm/sec) will be added to the apical chambers. Drug concentrations will be tested from the donor and receiver chambers at 0 and 2 h and compared to standard curves using LC/MS/MS. The apparent permeability is calculated as:

Papp(cm/sec)=(V/(A×Ci))×(Cf/T)

Where V is the volume of the receptor chamber, A is the area of the membrane insert, Ci is the initial dosing concentration, Cf is the final concentration of drug in the receiver well, and T is assay time in seconds.

MDCKII bidirectional assay was run in collaboration with Dr. Shaun Stauffer in Vanderbilt Institute of Chemical Biology (VICB) synthesis core who is a consultant on this project.

CYP inhibition: celastrol inhibits several CYP enzymes⁸. A broad panel of CYP enzymes have been examined, including 1A2, 2B6, 2D6, 2C8, 2C9, 2C19 and 3A4 for inhibition by celastrol and tiripentol in collaboration with Q² solutions. The data indicate celastrol on CYP inhibition will not prevent the compound from CNS drug development because the _{IC50} of celastrol for all the CYPs is at least 20 folds higher than its efficacious brain concentration (FIG. 2). Stiripentol, the approved drug for Dravet syndrome, has been reported to inhibit CYPs including 1A2, 2C9, 2C19, 2D6 and 3A4 with inhibition constant values at or slightly higher than its therapeutic concentrations³⁰ while our data indicates it is a potent inhibitor for 2C19 and 1A2 (Table 1). In conclusion, the effect of CYP inhibition will not prevent celastrol from drug development.

Oral formulation of celastrol: It has been reported that celastrol has oral bioavailability of 17.1% in rats³⁷ and lipid nanospheres could enhance oral bioavailability of celastrol to 30.01%³⁸. We have identified an oral bioavailability of 7.8% for celastrol in rats when administered in PEG400/saline/EtOH50/30/20 and an oral bioavailability of 20% for celastrol in mice when administered in suspension with a 20% hydroxypropyl-beta-cyclodextrin (HBPCD) 80% water (W/V) vehicle. This suggests a better bioavailability (30-50%) may be achievable via administration of solution doses using other vehicles not yet evaluated (e.g., lipid excipients such as Labrasol) and/or via reduction of particle size via milling. Evaluation will be initiated with lipid nanospheres for oral formulation.

Prospective Study of Pharmacodynamics and Pharmacokinetics and in Vivo Efficacy Via IP and Oral Gavage.

Therapeutic levels of the compound in brain and in blood indicate the EC₅₀s and/or IC₅₀s can be covered with IP dosing in pivotal mouse PD studies. Determination of the levels of the compound and its metabolites in brain tissue (somatosensory cortex in the forebrain), blood at the given dosages and the given time points as well as proteomics profiling of altered biochemical substrates at the given range of dosages will be conducted.

Experimental Methodology and Analysis:

Mice will be dosed via IP (0.1/kg-3 mg/kg) or oral gavage (0.5/kg-5 mg/kg) based on our preliminary data. Blood samples for drug bioanalysis will be collected in EDTA plasma tubes, immediately centrifuged, and the plasma fraction frozen at −80 C until analysis. Whole brains will also be frozen until analysis. Plasma and brain homogenate (prepared by bead beater in 70% isopropanol) concentrations of celastrol will be determined via comparison to standard curves prepared with control plasma/brain homogenate spiked with varying dilutions of test compound. Study samples, standards, and quality control samples will be precipitated with acetonitrile containing an internal standard, centrifuging the samples, and then injecting the supernatant onto a reverse phase LC/MS/MS system. Assay performance will be checked with retention time, peak shape, and quality control samples. The free drug concentration from animal studies will be calculated by multiplying the in vitro plasma free fraction (f_{unbound}) by the determined plasma concentrations. Image proteomics may also be used to determine the distribution pattern of celastrol in brain as well as other organs if necessary. ITRAQ and SILAC will be used to profile the broad biochemical changes in mouse cortex and cells treated with or without celastrol and will validate the key changes with antibody by Western blot. GABA_{A} receptor subunits, AKT and neuronal survival signaling molecules, heat shock protein and synaptic scaffold proteins will be the focus.

Prospective Study of the Validation of the Compound in Gabrg2^{+/Q390X} Model with Negative and Positive Controls and Benchmark Against Stiripentol.

The assay will include both biochemical, electrophysiological and neurobehavioral assessments including seizure severity and cognition. Demonstration if cortical neurodegeneration and synaptic injury in Gabrg2^{+/Q390X} mice were attenuated, demonstration if GABAergic neurotransmission was increased while EEG abnormality and seizure severity was reduced in Gabrg2^{+/Q390X} mice and demonstration if the impaired learning and memory in Gabrg2^{+/Q390X} mice was improved will be conducted.

Experimental Methodology and Analysis:

Established protocols for histology and immunohistochemistry to determine neurodegeneration in the mouse cortex will be used. The GABA_{A} receptor subunit expression will be determined especially γ2 subunits after celastrol treatment. Distribution of the subunits in somata and dendrites and synapses and the expression of the active form of caspase 3 as it represents a marker for apoptosis and celastrol could reduce the expression of caspase 3 in Gabrg2^{+/Q390X} mice will also be determined (FIG. 17). Biochemical and electrophysiological characterizations will focus on GABA_{A} receptor expression, heat shock chaperone protein profiling, neuroprotective AKT signaling, synaptic injury and scaffolding molecule expression and GABAergic neurotransmission. For synaptic scaffold proteins, we will determine gephyrin, collybistin, synaptogamin 1 and neuroligin II as they are the key molecules in inhibitory synapses and our preliminary data have indicated that they were reduced in synaptosomes (FIG. 18).

Protocols have been developed for synaptosome isolation, mouse brain preparations and immunohistochemistry. Proposed antibodies have been validated and are specific to the antigens we are testing. As to cortical neurodegeneration and synaptic injury, neurodegeneration and synaptic injury in Gabrg2^{+/Q390X} mice has been demonstrated; it is unknown if there is any neurodegeneration in the Scn1a^{+/-} mice. However, it has been reported that SCN1A mutation may play a direct role in encephalopathy in addition to seizures. Preliminary data indicates synaptic injury as evidenced by reduced synaptic scaffold proteins like gephyrin in Scn1a^{+/-} mice. The Scn1a^{+/-} mouse model will be the focus of EEG recordings because it represents ˜80% of Dravet syndrome but will focus on Gabrg2^{+/Q390X} mice for in vitro cell based study because of the mutant protein aggregation.

Based on our preliminary data and the power analysis for statistics, we will use 4 pairs of mice for immunohistochemistry and GABAergic neurotransmission and 10-12 pairs of mice for EEG analysis and Barnes maze test for cognition.

Discussion

A novel compound has been identified that can attenuate the severity of the seizures and comorbidities in a novel severe epilepsy mouse model Gabrg2+/Q390X mice. This mouse model has been characterized in more detail and the novel pathophysiology identified, the accumulation of the mutant subunit protein worsening the severe seizure phenotype. Thus, it is likely the mechanism of how celastrol reduces the epilepsy severity in this mouse model has been identified. Because GABRG2(Q390X) is only identified in a few pedigrees, compound has been tested in other epilepsy mouse models including Scn1a+/−, a mouse model for ˜80% of Dravet syndrome. Importantly, we have identified that celastrol could upregulate GABAA receptor at the cell surface and the total levels in both mouse models. This may explain its effect of reducing seizures. This will not only lay critical groundwork for advancing celastrol as a novel drug to treat epilepsy and many other neurodegenerative diseases with overlapping pathophysiology. This contribution will be seminal for developing more mechanism-based therapies with similar structures and targeting similar mechanisms for epilepsy as well as for many other neurological disorders. the disease phenotype of Gabrg2+/− mice is less severe than Gabrg2+/Q390X mice because the moderate amount of increase in the wild-type γ2 subunits. Improvement in behavioral seizures, GABAergic neurotransmission, learning and memory will also indicate advancing of celastrol into drug development. The surface increase of γ2 subunits in the mouse cortex and the frequency of seizures as criteria for advancing the compound to next phase. We have already demonstrated that celastrol (0.3 mg/kg, ip) could increase surface γ2 subunits to over 40-50% compared with the mutant mice treated with vehicle. We have demonstrated the seizure reduction from celastrol at 1 and 5 mg/kg via oral gavage but will determine the dosage via oral gavage to achieve at least ˜25% of increase of γ2 subunit expression and reduction of SWDs to less than 2/hr in C57BL/6J Gabrg2+/Q390 mice.

Prospective Study of Oral Formulation

An enabling oral formulation (e.g. suspension or solution) will be used to determine oral PK and model oral doses that will provide effective drug exposures. Most chronic therapies for humans demand oral delivery for compliance and ease of administration, and this is also how celastrol has been administered in Chinese herbal medicine. Typical formulation strategies that will enable rodent pharmacology and preclinical testing will be employed, such as aqueous suspensions containing surfactants, and aqueous-based solutions containing co-solvents such as polyethylene glycol and ethanol. Oral gavage at 3 mg/kg for 2 weeks has been demonstrated as increasing seizure threshold and reducing seizure activity. The brain concentration was 121 nM (2 hrs after oral gavage). The brain concentration and the in vivo efficacy are encouraging.

A 30.01% bioavailability has been reported with lipid nanospheres³⁸. We have identified an bioavailability of ˜20% and will try to get 30-50% in order to develop a long-term solution to generate appropriate exposure with oral dosing regimens in humans. In order to do this, we need to try a formulation strategy that will significantly improve its aqueous solubility. The cut-off bioavailability is 20% for further study with orally delivered celastrol because of the already identified bioavailability of 20%.

Intermittent dosing: It is clear that intermittent dosing regimen (FIG. 4A) is efficacious and well tolerated. However, the length of minimal dosing and maximal drug holiday is unknown. The dosing regimen of orally delivered celastrol will be determined. The data of intermittent dosing from IP injection will be used to guide our oral dosing regimen, and GABA_{A} receptor expression, GABAergic neurotransmission, and synaptic scaffold molecules as biological readouts and in vivo efficacy will be used to validate the effect of oral dosing. drug

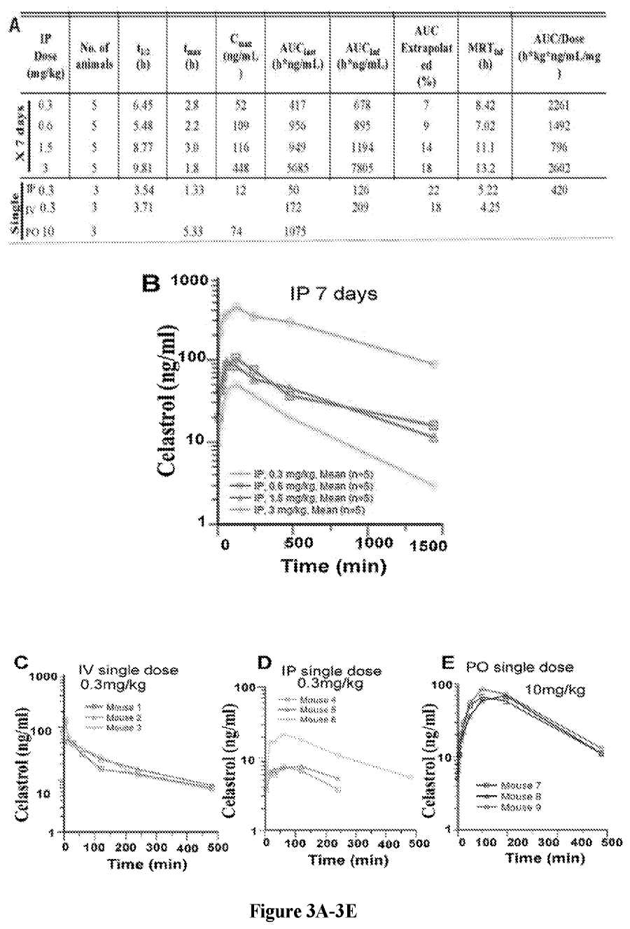

concentrations can be routinely quantitated down to 1 ng/mL with LC/MS/MS, thus allowing pharmacokinetics and brain drug levels to be assessed from animal studies. We will use the same approaches used in R21 phase for R33 phase. We had substantially characterized The efficacy of celastrol at 0.3 mg/kg (IP) has been substantially characterized. The therapeutic dosages and the levels of the compound in the brain and the plasma with oral delivered celastrol will be refined, focusing on 3 mg/kg for oral formulations and be guided by PK data (FIGS. 3E and 4B).

Survival and seizure activity in Scn1a^{+/-} mice after off celastrol treatment for 1 month (0.3 mg/kg 14 d on and 1 month off) has been tested. Compared with the littermates without celastrol treatment, the celastrol treated mice had increased survival, delayed seizure onset and shortened seizure duration and reduced seizure severity after PTZ injection. This suggests celastrol could be dosed intermittently. Intermittent dosing will greatly increase compliance and reduce side effects for long-term treatment. However, it is necessary to determine the duration the efficacy lasts after certain doses and the duration it takes to recover to baseline.

We will use the same experimental approaches we have established in our preliminary study for measurements in behaviors, EEGs and seizures. Vehicle will be treated as negative control and the wild-type as positive control. Other compounds known with similar pharmacological effect, we will include the compound(s). We have included another herbal derivative curcumin in our study because Curcumin has been reported to have overlapping effect with celastrol³³ and improve memory in Alzheimer's mice^{6,7,29}. Both compounds activate PI3K/AKT pathway in vitro. However, curcumin was impermeable in MDCK II bidirectional assay while celastrol Papp was high (A-B Mean Papp 56.48). Stiripentol was measured but it showed poor signal to noise ratio. Because diazepam enhance GABA_{A} receptor function and has been widely used for epilepsy including Dravet syndrome, we will compare the in vivo efficacy of celastrol, diazepam and stiripentol alone or in combination to determine which group has better seizure control as well as improvement in cognition.

University to calculate the sample sizes for survival, seizure induction test and seizure frequency measurements. In each mouse strain, we will compare two genotype groups, the wild-type vs the mutant. In each genotype, we will compare the drug treated vs vehicle treated groups. We will compare the groups in mortality rate, frequency of animals reached generalized tonic clonic seizures (GTCS) which is stage 4-5 based on Racine scale. We will also compare the frequency of spontaneous GTCS, myoclonic jerks and the frequency and duration of spike wave discharges (SWDs). Based on our preliminary data, we estimated the sample sizes required to achieve 80% power with two-sided Type I error rate of 0.05 at several effect sizes, using Chi-squared tests (for mortality rate and seizure grade based on Racine scale) and two-sample t-test (for EEGs). We performed the estimations with Stata 14 software. We also recruited Dr. Du to join our study (Please see the letter of support).

By successful completion of these studies, we have refined a novel compound for treating epilepsy via a more feasible and safe way of delivery. We have validated the effect of the compound in a more rigorous study design. We have validated the compound with both negative and positive controls. We have advanced celastrol as a candidate for preclinical development.

Celastrol is a very promising compound for being advanced for a CNS drug. It is highly brain permeable and possesses multiple favorable pharmaceutical properties. The safety margin is at least 5 folds of efficacious dose and it is well tolerated with intermittent dosing regimen. Because of the low doses proposed, previously reported toxicity with doses for tumor is likely unrelated. The long-term use in traditional Chinese medicine also suggests its time-tested safety. The identified bioavailability is 20% and could be further improved to 30-50%. New compounds can be created around the parent compound with reduced toxicity and enhanced permeability based on previous findings^{19,31}. Furthermore, celastrol may have a broad application for multiple diseases based on its multiple molecular actions and correct dosing. However, in epilepsy, the primarily involved biological pathways are enhanced GABAergic neurotransmission via upregulated GABA_{A} receptors and reduced the neuronal/synaptic injury. Further investigation can include determination if other ion channel or non-ion channel proteins are changed, and the possible impact on long-term biologic function.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

¹ A. C. Allison, et al., “Celastrol, a potent antioxidant and anti-inflammatory drug, as a possible treatment for Alzheimer's disease,” Prog. Neuropsychopharmacol. Biol. Psychiatry 25(7), 1341 (2001).
² F. M. Arain, K. L. Boyd, and M. J. Gallagher, “Decreased viability and absence-like epilepsy in mice lacking or deficient in the GABAA receptor alpha1 subunit,” Epilepsia 53(8), e161-e165 (2012).
³ G. L. Carvill, et al., “GABRA1 and STXBP1: Novel genetic causes of Dravet syndrome,” Neurology (2014).
⁴ L. A. Harkin, et al., “Truncation of the GABA(A)-receptor gamma2 subunit in a family with generalized epilepsy with febrile seizures plus,” Am. J. Hum. Genet. 70(2), 530 (2002).
⁵ S. Hirose, et al., “SCN1A testing for epilepsy: application in clinical practice,” Epilepsia 54(5), 946 (2013).
⁶ J. B. Hoppe, et al., “The curry spice curcumin attenuates beta-amyloid-induced toxicity through beta-catenin and PI3K signaling in rat organotypic hippocampal slice culture,” Neurol. Res. 35(8), 857 (2013).
⁷ J. B. Hoppe, et al., “Curcumin protects organotypic hippocampal slice cultures from Abeta1-42-induced synaptic toxicity,” Toxicol. In Vitro 27(8), 2325 (2013).
⁸ C. Jin, et al., “Inhibitory mechanisms of celastrol on human liver cytochrome P450 1A2, 2C19, 2D6, 2E1 and 3A4,” Xenobiotica 45(7), 571 (2015).
⁹ Shen W Zhou C Xu D Macdonald R L Kang J Q, “The Human Epilepsy Mutation GABRG2(Q390X) Causes Chronic Subunit Accumulation and Neurodegeneration,” in 2015).
¹⁰ J. Q. Kang and R. L. Macdonald, “The GABAA receptor gamma2 subunit R43Q mutation linked to childhood absence epilepsy and febrile seizures causes retention of alpha1beta2gamma2S receptors in the endoplasmic reticulum,” J. Neurosci. 24(40), 8672 (2004).
¹¹ J. Q. Kang and R. L. Macdonald, “Molecular Pathogenic Basis for GABRG2 Mutations Associated With a Spectrum of Epilepsy Syndromes, From Generalized Absence Epilepsy to Dravet Syndrome,” JAMA Neurol. (2016).
¹² J. Q. Kang, et al., “Slow degradation and aggregation in vitro of mutant GABAA receptor gamma2(Q351X) subunits associated with epilepsy,” J. Neurosci. 30(41), 13895 (2010).
¹³ J. Q. Kang, et al., “Slow degradation and aggregation in vitro of mutant GABAA receptor gamma2(Q351X) subunits associated with epilepsy,” J. Neurosci. 30(41), 13895 (2010).
¹⁴ J. Q. Kang, W. Shen, and R. L. Macdonald, “The GABRG2 mutation, Q351X, associated with generalized epilepsy with febrile seizures plus, has both loss of function and dominant-negative suppression,” J. Neurosci. 29(9), 2845 (2009).
¹⁵ J. Q. Kang, W. Shen, and R. L. Macdonald, “Trafficking-deficient mutant GABRG2 subunit amount may modify epilepsy phenotype,” Ann. Neurol. 74(4), 547 (2013).
¹⁶ J. Q. Kang, W. Shen, and R. L. Macdonald, “Trafficking-deficient mutant GABRG2 subunit amount may modify epilepsy phenotype,” Ann. Neurol. 74(4), 547 (2013).
¹⁷ J. Q. Kang, et al., “The human epilepsy mutation GABRG2(Q390X) causes chronic subunit accumulation and neurodegeneration,” Nat. Neurosci. 18(7), 988 (2015).
¹⁸ J. Q. Kang, et al., “The human epilepsy mutation GABRG2(Q390X) causes chronic subunit accumulation and neurodegeneration,” Nat. Neurosci. (2015).
¹⁹ J. Q. Kang, et al., “The human epilepsy mutation GABRG2(Q390X) causes chronic subunit accumulation and neurodegeneration,” Nat. Neurosci. 18(7), 988 (2015).
²⁰ Y. O. Kim, et al., “Do mutations in SCN1B cause Dravet syndrome?,” Epilepsy Res. 103(1), 97 (2013).
²¹ K. Kobow, et al., “Finding a better drug for epilepsy: antiepileptogenesis targets,” Epilepsia 53(11), 1868 (2012).
²² S. C. Landis, et al., “A call for transparent reporting to optimize the predictive value of preclinical research,” Nature 490(7419), 187 (2012).
²³ C. Marini, et al., “The genetics of Dravet syndrome,” Epilepsia 52 Suppl 2, 24 (2011).
²⁴ M. H. Meisler and J. A. Keamey, “Sodium channel mutations in epilepsy and other neurological disorders,” J. Clin. Invest 115(8), 2010 (2005).
²⁵ A. M. Mistry, et al., “Strain- and age-dependent hippocampal neuron sodium currents correlate with epilepsy severity in Dravet syndrome mice,” Neurobiol. Dis. 65, 1 (2014).
²⁶ S. E. Ong, G. Mittler, and M. Mann, “Identifying and quantifying in vivo methylation sites by heavy methyl SILAC,” Nat. Methods 1(2), 119 (2004).
²⁷ X. K. Ouyang, et al., “Development and validation of a liquid chromatography coupled with atmospheric-pressure chemical ionization ion trap mass spectrometric method for the simultaneous determination of triptolide, tripdiolide, and tripterine in human serum,” J. Anal. Toxicol. 32(9), 737 (2008).
²⁸ D. Paris, et al., “Reduction of beta-amyloid pathology by celastrol in a transgenic mouse model of Alzheimer's disease,” J. Neuroinflammation. 7, 17 (2010).
²⁹ R. Patil, et al., “Curcumin Targeted, Polymalic Acid-Based MRI Contrast Agent for the Detection of Abeta Plaques in Alzheimer's Disease,” Macromol. Biosci. 15(9), 1212 (2015).
³⁰ X. Shi, et al., “Clinical spectrum of SCN2A mutations,” Brain Dev. 34(7), 541 (2012).
³¹ A. Tran, et al., “Influence of stiripentol on cytochrome P450-mediated metabolic pathways in humans: in vitro and in vivo comparison and calculation of in vivo inhibition constants,” Clin. Pharmacol. Ther. 62(5), 490 (1997).
³² T. A. Warner, et al., “DIfferential molecular and behavioral alterations in mouse models of GABRG2 haploinsufficiency versus dominant negative mutations associated with human epilepsy,” Hum. Mol. Genet. (2016).
³³ S. Weisberg, R. Leibel, and D. V. Tortoriello, “Proteasome inhibitors, including curcumin, improve pancreatic beta-cell function and insulin sensitivity in diabetic mice,” Nutr. Diabetes 6, e205 (2016).
³⁴ G. Xia, et al., “Altered GABA_{A} receptor expression in brainstem nuclei and SUDEP in Gabrg2(+/Q390X) mice associated with epileptic encephalopathy,” Epilepsy Res. 123, 50 (2016).
³⁵ L. Yang, et al., “Celastrol attenuates inflammatory and neuropathic pain mediated by cannabinoid receptor type 2,” Int. J. Mol. Sci. 15(8), 13637 (2014).
³⁶ F. H. Yu, et al., “Reduced sodium current in GABAergic interneurons in a mouse model of severe myoclonic epilepsy in infancy,” Nat. Neurosci. 9(9), 1142 (2006).
³⁷ J. Zhang, et al., “Oral bioavailability and gender-related pharmacokinetics of celastrol following administration of pure celastrol and its related tablets in rats,” J. Ethnopharmacol. 144(1), 195 (2012).
³⁸ X. Zhang, et al., “Enhancement of oral bioavailability of tripterine through lipid nanospheres: preparation, characterization, and absorption evaluation,” J. Pharm. Sci. 103(6), 1711 (2014).
³⁹ C. Zhou, et al., “Altered cortical GABAA receptor composition, physiology, and endocytosis in a mouse model of a human genetic absence epilepsy syndrome,” J. Biol. Chem. 288(29), 21458 (2013).
⁴⁰ C. Zhou, et al., “Hypoxia-induced neonatal seizures diminish silent synapses and long-term potentiation in hippocampal CA1 neurons,” J. Neurosci. 31(50), 18211 (2011).

It will be understood that various details of the presently disclosed subject matter can be changed without departing from the scope of the subject matter disclosed herein. Furthermore, the foregoing description is for the purpose of illustration only, and not for the purpose of limitation.

1. A method of treating a condition with reduced GABA_{A}, the method comprising:
administering celastrol to a subject in need of treatment for a condition with reduced GABA_{A}.

2. The method of claim 1, further comprising administering diazepam.

3. The method of claim 1, wherein the celastrol is administered orally, intraperitoneally, or intravenously.

4. The method of claim 3, wherein the celastrol is administered intraperitoneally in the range of 0.1 mg/kg to 0.5 mg/kg.

5. The method of claim 3, wherein the celastrol is administered orally at a daily dose of about 5-10 mg.

6. The method of claim 1, wherein the condition is epilepsy.

7. The method claim 6 wherein the epilepsy is selected from Dravet syndrome, primary epilepsy or secondary epilepsy.

8. The method of claim 1, wherein the subject is an animal subject, celastrol is provided in an animal food product, and the administering comprises feeding the animal subject the animal food product.

9. A method of treating a condition selected from neurological diseases, central nervous system (CNS) disorders, and inflammatory diseases, the method comprising:
administering celastrol to a subject in need of treatment for a neurological disease, a CNS disorder, or an inflammatory disease.

10. The method of claim 9, wherein the condition is selected from encephalitis, Alzheimer's, Parkinson's or Huntington's.

11. The method of claim 9, wherein the treatment delays seizure onset, shortens seizure duration, or reduces seizure severity.

12. The method of claim 9, wherein the administering comprises intermittent dosing.

13. The method of claim 12, wherein the celastrol is administered at 0.1 mg/kg to about 20 mg/kg.

14. The method of claim 13, wherein the celastrol is administered at 0.1 mg/kg to about 1 mg/kg.

15. A method of treating a condition with reduced GABA_{A}, or a condition selected from neurological diseases, central nervous system (CNS) disorders, and inflammatory diseases, the method comprising: administering a derivative of celastrol to a subject in need of treatment, wherein the derivative of celastrol is selected from

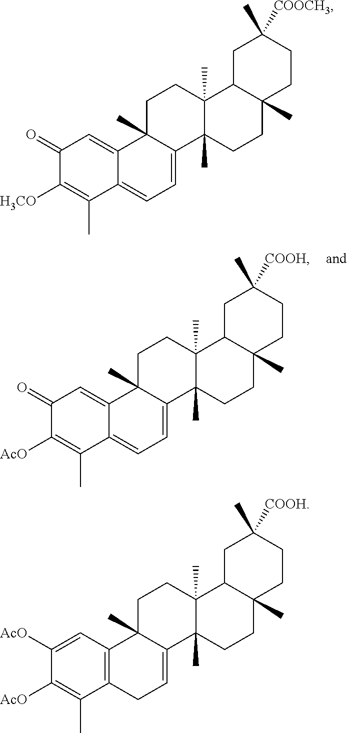 [SMILES: COC(=O)[C@]1(C)CC[C@]2(C)CC[C@]3(C)C4=CC=C5C(=CC(=O)C(OC)=C5C)[C@]4(C)CC[C@@]3(C)C2C1 ; CC(=O)OC1=C(C)C2=CC=C3[C@@](C)(CC[C@@]4(C)C5C[C@](C)(C(=O)O)CC[C@]5(C)CC[C@]34C)C2=CC1=O ; CC(=O)Oc1cc2c(c(C)c1OC(C)=O)CC=C1[C@@]2(C)CC[C@@]2(C)C3C[C@](C)(C(=O)O)CC[C@]3(C)CC[C@]12C]

16. The method of claim 1, wherein the celastrol is administered orally as a suspension or solution.

17. The method of claim 16, wherein the celastrol is provided as a lipid nanoparticle suspension.

18. The method of claim 16, wherein the celastrol is first milled to reduce particle size.

19. The method of claim 18, wherein the final particle size is about 65-85 nm.

20. The method of claim 16, wherein the celastrol is provided in a 20% suspension hydroxypropyl-beta-cyclodextrin (HPBCD) 80% w/v water vehicle.
